(19) United States
(12) Patent Application Publication
(10) Pub. No.: US 2007/0207946 A1
(43) Pub. Date: Sep. 6, 2007

(54) Dipeptidyl peptidase IV inhibitors and their uses for lowering blood pressure levels

(76) Inventors: Andrew J. Pospisilik, Vancouver (CA); Hans Ulrich Demuth, Halle (DE); Konrad Glund, Halle (DE); Matthias Hoffmann, Weingelsdorf (DE); Christopher H. S. McIntosh, Vancouver (CA); Ray A. Pederson, Vancouver (CA)

Correspondence Address: OSI PHARMACEUTICALS, INC., 41 PINELAWN ROAD, MELVILLE, NY, 11747, US

(21) Appl. No.: 11/800,576

(22) Filed: May 3, 2007

Related U.S. Application Data
(63) Continuation of application No. 11/377,530, filed on Mar. 16, 2006.
(63) Continuation of application No. 10/970,526, filed on Oct. 21, 2004, now abandoned.
Application No. 10/970,526 is a continuation of application No. 10/200,919, filed on Jul. 23, 2002, now abandoned.
Application No. 10/200,919 is a continuation-in-part of application No. 09/932,546, filed on Aug. 17, 2001, now abandoned.

(51) Int. Cl. A61K 31/40; A61K 31/425; A61K 38/00

(52) U.S. Cl. 514/2; 514/371; 514/423

(57) ABSTRACT

The present invention provides new uses of DPIV-inhibitors of the present invention, and their corresponding pharmaceutically acceptable acid addition salt forms, for lowering blood pressure levels.

### RELATED APPLICATIONS

This application is a continuation of U.S. application Ser. No. 10/970,526, filed Oct. 21, 2004, which is a continuation of U.S. application Ser. No. 10/200,919, filed Jul. 23, 2002 which is a continuation in part of U.S. application Ser. No. 09/932,546 filed Aug. 17, 2001 which claims the benefit from U.S. application Ser. No. 09/155,833, filed Oct. 6, 1998, now U.S. Pat. No. 6,303,661, all of which are incorporated by reference in their entirety.

### FIELD OF THE INVENTION

The present invention relates to inhibitors of dipeptidyl peptidase IV and dipeptidyl peptidase IV-like enzyme activity and, more particularly, pharmaceutical compositions containing said compounds, and the use of said compounds for lowering blood pressure levels in mammals and related disorders.

### BACKGROUND ART

Dipeptidyl peptidase IV (DPIV) is a serine protease which cleaves N-terminal dipeptides from a peptide chain containing, preferably, a proline residue in the penultimate position. Although the biological role of DPIV in mammalian systems has not been completely established, it is believed to play an important role in neuropeptide metabolism, T-cell activation and the entry of HIV into lymphoid cells.

The present invention provides a new use of DPIV-inhibitors for the prophylaxis and treatment of conditions mediated by inhibition of DPIV and DPIV-like enzymes, in particular for lowering blood pressure levels and related disorders, and pharmaceutical compositions e.g. useful in inhibiting DPIV and DPIV-like enzymes and a method of inhibiting said enzyme activity.

This invention relates to a method of treatment, in particular to a method for lowering blood pressure levels in mammals and to compounds and compositions for use in such method. Dipeptidyl peptidase IV (DPIV; EC 3.4.14.5; CD26) is a post-proline (to a lesser extent post-alanine, post-serine or post-glycine) cleaving serine protease that is expressed on a number of tissues, including epithelial cells and leukocyte subsets. Furthermore, it is a membrane-associated ectopeptidase which exhibits its activity in its extracellular domain.

Examples of low molecular weight dipeptidyl peptidase IV inhibitors are agents such as tetrahydroisoquinolin-3-carboxamide derivatives, N-substituted 2-cyanopyroles and -pyrrolidines, N-(N′-substituted glycyl)-2-cyanopyrrolidines, N-(substituted glycyl)-thiazolidines, N-(substituted glycyl)-4-cyanothiazolidines, amino-acyl-borono-prolyl-inhibitors, cyclopropyl-fused pyrrolidines and heterocyclic compounds. Inhibitors of dipeptidyl peptidase IV are described in U.S. Pat. No. 6,380,398, U.S. Pat. No. 6,011,155; U.S. Pat. No. 6,107,317; U.S. Pat. No. 6,110,949; U.S. Pat. No. 6,124,305; U.S. Pat. No. 6,172,081; WO 95/15309, WO 99/61431, WO 99/67278, WO 99/67279, DE 198 34 591, WO 97/40832, DE 196 16 486 C 2, WO 98/19998, WO 00/07617, WO 99/38501, WO 99/46272, WO 99/38501, WO 01/68603, WO 01/40180, WO 01/81337, WO 01/81304, WO 01/55105, WO 02/02560 and WO 02/14271, the teachings of which are herein incorporated by reference in their entirety, especially concerning these inhibitors, their definition, uses and their production.

The term DPIV-like enzymes relates to structurally and/or functionally DPIV/CD26-related enzyme proteins (Sedo & Malik, Dipeptidyl peptidase IV-like molecules: homologous proteins or homologous activities? Biochimica et Biophysica Acta 2001, 36506: 1-10). In essence, this small group of enzymes has evolved during evolution to release H-Xaa-Pro-Dipeptides and H-Xaa-Ala-Dipeptides from N-terminus of oligo- or polypeptides. They show the common feature that they accommodate in the Pro-position also Ala, Ser, Thr and other amino acids with small hydrophobic side-chains as, Gly or Val. The hydrolytic efficacy is ranked Pro>Ala>>Ser, Thr>>Gly, Val. Same proteins have been only available in such small quantities, that only the post-Pro or post-Ala cleavage could be established. While the proteins: DPIV, DP II, FAPα (Seprase), DP 6, DP 8 and DP 9 are structurally related and show a high sequence homology, attractin is an extraordinary functional DPIV-like enzyme, characterized by a similar activity and inhibitory pattern.

Further DPIV-like enzymes are disclosed in WO 01/19866, WO 02/04610, WO 02/34900 and WO02/31134. WO 01/19866 discloses novel human dipeptidyl aminopeptidase (DPP8) with structural and functional similarities to DPIV and fibroblast activation protein (FAP). WO 02/34900 discloses a novel dipeptidyl peptidase 9 (DPP9) with significant homology with the amino acid sequences of DPIV and DPP8. WO 02/31134 discloses three DPIV-like enzymes, DPRP1, DPRP2 and DPRP3. Sequence analysis revealed, that DPRP1 is identical to DPP8, as disclosed in WO 01/19866, that DPRP2 is identical to DPP9 and that DPRP3 is identical to KIAA1492 as disclosed in WO 02/04610.

High blood pressure (hypertension) is generally a symptomless condition in which abnormally high pressure in the arteries increases the risk of problems such as stroke, aneurysm, heart failure, heart attack, and kidney damage. To many people, the word hypertension suggests excessive tension, nervousness, or stress. In medical terms, however, hypertension refers to a condition of elevated blood pressure, regardless of the cause. It has been called “the silent killer” because it usually doesn't cause symptoms for many years—until a vital organ is damaged. High blood pressure is defined as a systolic pressure at rest that averages 140 mm Hg or more, a diastolic pressure at rest that averages 90 mm Hg or more, or both. In high blood pressure, usually both the systolic and the diastolic pressures are elevated.

As a secondary effect of diabetes mellitus, the nerves that control blood pressure and digestive processes become damaged. This results in swings in blood pressure; swallowing difficulties and altered gastrointestinal function, with bouts of diarrhea. Furthermore, as a secondary effect of diabetes mellitus, atherosclerotic plaques build up and block large or medium-sized arteries in the heart, brain, legs, and penis. The walls of small blood vessels are damaged so that the vessels do not transfer oxygen normally and may leak.

Further definitions and a classification of high blood pressure is given in The Merck Manual of Medical Information-Home Edition, Merck & Co., 2000. When a person's systolic and diastolic pressures fall into different categories, the higher category is used to classify blood pressure. For instance, 160/92 is classified as stage 2 hypertension, and 180/120 is classified as stage 4 hypertension. The optimal blood pressure for minimizing the risk of cardiovascular problems is below 120/80 mm Hg. However, unusually low readings must be evaluated.

| | Systolic blood | Diastolic blood |
|---|---|---|
| Category | pressure | pressure |
| Normal blood pressure | Below 130 mmHg | Below 85 mmHg |
| High normal blood pressure | 130-139 | 85-89 |
| Stage 1 (mild) hypertension | 140-159 | 90-99 |
| Stage 2 (moderate) hypertension | 160-179 | 100-109 |
| Stage 3 (severe) hypertension | 180-209 | 110-119 |
| Stage 4 (very severe) | 210 or higher | 120 or higher |
| hypertension | | |

If a person has high blood pressure that's severe or long-standing and untreated, symptoms such as headache, fatigue, nausea, vomiting, shortness of breath, restlessness, and blurred vision occur because of damage to the brain, eyes, heart, and kidneys. Occasionally, people with severe high blood pressure develop drowsiness and even coma caused by brain swelling. This condition, called hypertensive encephalopathy, requires emergency treatment.

Untreated high blood pressure increases a person's risk of developing heart disease (such as heart failure or heart attack), kidney failure, and stroke at an early age. High blood pressure is the most important risk factor for stroke. It's also one of the three major risk factors for heart attack (myocardial infarction) that a person can do something about; the other two are smoking and high blood cholesterol levels.

### SUMMARY OF THE INVENTION

The present invention provides new uses of DPIV-inhibitors of formulas 1 to 12, and their corresponding pharmaceutically acceptable acid addition salt forms for lowering blood pressure levels or related disorders in mammals.

Reduced expression of the ectopeptidase DPIV and lack of DPIV-like activity in mutant F344 rats lacking DPIV enzymic activity and expression results in a lowered blood pressure. Mutant F344 substrains lacking DPIV enzymic activity and wild-type-like F344 were tested. Chronic intragastric infusion of isoleucyl cyano pyrrolidine TFA and isoleucyl thiazolidine fumarate via osmotic minipumps over two weeks dose-dependently reduced the blood pressure of the rats. Thus, blood pressure is reduced by chronic treatment using different DPIV Inhibitors (isoleucyl thiazolidine fumarate; isoleucyl cyano pyrrolidine TFA) suggesting protective-like class effects by the two different DPIV-inhibitors/ligands. Possibly, isoleucyl thiazolidine fumarate and isoleucyl cyano pyrrolidine TFA protect from high blood pressure via increased levels of DPIV substrates, which indirectly mediate corresponding effects.

The present invention relates to a novel method in which reduction of the activity of the enzyme Dipeptidyl Peptidase (DPIV or CD26), or of DPIV-like enzyme activity, in the blood of mammals by specific enzyme effectors will result in a reduced degradation of the endogenous, or exogenously administrated, insulinotropic peptides (incretins), Gastric Inhibitory Polypeptide/Glucose-dependent Insulinotropic Polypeptide 1-42 (GIP₁₋₄₂) and Glucagon-like Peptide-1 7-36 amide (GLP-1₇₋₃₆) (or analogs of these peptides). The decrease in concentration of these peptides or their analogs, resulting from degradation by DPIV and DPIV-like enzymes, will be thus be reduced or delayed.

As a consequence of the enhanced stability of the endogenous, or exogenously administered, incretins or their analogs, caused by a reduction in DPIV-activity, their insulinotropic effects are enhanced, resulting in a potentate stimulation of insulin secretion from the pancreatic islets of Langerhans, and more rapid removal of glucose from the blood. As a result, glucose tolerance is improved.

As a consequence, metabolic abnormalities associated with Diabetes mellitus, including abnormalities of carbohydrate and lipid metabolism, glucosuria and diabetic ketoacidosis, and chronic alterations such as microvascular and macrovascular disease, polyneuropathy and diabetic retinopathy, which are the consequence of prolonged, elevated circulating glucose concentrations, are prevented or alleviated and in particular high blood pressure levels are reduced.

The present invention is a new approach to lowering elevated concentrations of blood glucose and elevated blood pressure levels. It is simple, commercially useful, and is suitable to be used in the therapy, especially of human diseases, which are caused by elevated or extraordinary blood glucose and/or blood pressure levels.

### BRIEF DESCRIPTION OF DRAWINGS

Further understanding of the present invention may be had by reference to the accompanying drawings wherein:

FIG. 1 shows MALDI-TOF-analysis of the DPIV-catalyzed hydrolysis of GIP₁₋₄₂ (a) and GLP-₇₋₃₆ and their inhibition by isoleucyl thiazolidine (b).

FIG. 2 shows HPLC-analysis of the serum presence of GLP-1 metabolites in presence of the DPIV inhibitor isoleucyl thiazolidine in vivo.

FIG. 3 shows influence of the DPIV-inhibitor isoleucyl thiazolidine on different blood parameter of the i.d.-glucose-stimulated rat.

FIG. 4 shows influence of chronic oral treatment of fatty (fa/fa) VDF Zucker rats by the DPIV-inhibitor isoleucyl thiazolidine on the fasting blood glucose during 12 weeks of drug application.

FIG. 5 Influence of chronic treatment of fatty (fa/fa) VDF Zucker rats by the DPIV-inhibitor isoleucyl thiazolidine on the systolic blood pressure within 8 weeks of drug application (systolic blood pressure was measured using the tail-cuff procedure).

FIG. 6 shows the dose dependent lowering of blood glucose levels in diabetic Zucker rats following oral administration of 5 mg/kg, 15 mg/kg, 50 mg/kg b.w. glutaminyl pyrrolidine and placebo, respectively;

FIG. 7 shows the dose dependent lowering of blood glucose levels in diabetic Zucker rats following oral administration of 5 mg/kg, 15 mg/kg, 50 mg/kg b.w. glutaminyl thiazolidine and placebo, respectively;

FIG. 8 shows the chemical structure of pyroglutaminyl thiazolidine, the degradation product, found after oral administration of glutaminyl thiazolidine to Wistar rats; and

FIG. 9 shows the chromatogram of a rat plasma extract obtained after oral administration of glutaminyl thiazolidine to fatty Zucker rats. The peak at 2.95 min represents glutaminyl thiazolidine and the peak at 6.57 min represents pyroglutaminyl thiazolidine.

### DETAILED DESCRIPTION OF THE INVENTION

The aim of the present invention is a simple and new method to lower the level of blood glucose and/or blood pressure in which reduction in the activity of the enzyme dipeptidyl peptidase IV (DPIV or CD26) or of DPIV-like enzyme activity in the blood of mammals induced by effectors of the enzyme will lead to a reduced degradation of the endogenous (or exogenously administrated) insulinotropic peptides Gastric Inhibitory Polypeptide 1-42 (GIP₁₋₄₂) and Glucagon-Like Peptide Amide-1 7-36 (GLP-1₇₋₃₆) (or analogs of these peptides). The decrease in concentration of these peptides or their analogs, normally resulting from degradation by DPIV and DPIV-like enzymes, will thus be reduced or delayed.

The present invention is based on the striking finding that a reduction in the enzymatic activity of dipeptidyl peptidase IV (DPIV or CD26) or of DPIV-like enzyme activity in the body of mammals in vivo results in an improved glucose tolerance and in a reduction of high blood pressure.

We observed that:

1. Reduction of dipeptidyl peptidase IV (DPIV or CD26) or of DPIV-like enzyme activity leads to an increase in the stability of glucose-stimulated endogenously released or exogenously administrated incretins (or their analogs) with the consequence that the administration of effectors of DPIV or of DPIV-like proteins can be used to control the incretin degradation in the circulation.

2. The enhanced biological stability of the incretins (or their analogs) results in a modification of the insulin response.

3. The enhanced stability of the circulating incretins, caused by reduction of dipeptidyl peptidase IV (DPIV or CD26) or of DPIV-like enzyme, results in subsequent modification of insulin-induced glucose disposal, indicating that glucose tolerance can be improved by applying DPIV-effectors.

4. High blood pressure levels are reduced.

Accordingly, the invention concerns the use of effectors of dipeptidyl peptidase IV (DPIV) or of DPIV-like enzyme activity, for lowering of elevated blood glucose and/or blood pressure levels, such as those found in mammals demonstrating clinically inappropriate basal and post-prandial hyperglycemia. The use according to the invention is more specifically characterized by the administration of effectors of DPIV or of DPIV-like enzyme activity in the prevention or alleviation of pathological abnormalities of metabolism of mammals such as glucosuria, hyperlipidaemia, diabetic ketoacidosis, diabetic retinopathy and diabetes mellitus. In a further preferred embodiment, the invention concerns a method of lowering elevated blood glucose levels in mammals, such as those found in a mammal demonstrating clinically inappropriate basal and post-prandial hyperglycemia, comprising administering to a mammal in need of such treatment a therapeutically effective amount of an effector of dipeptidyl peptidase IV (DPIV) or of DPIV-like enzyme activity.

In another preferred embodiment, the invention concerns effectors of dipeptidyl peptidase IV (DPIV) or of DPIV-Iike enzyme activity for use in a method of lowering elevated blood glucose and/or blood pressure levels in mammals, such as those found in mammals demonstrating clinically inappropriate basal and post-prandial hyperglycemia.

The administered effectors of DPIV and DPIV-like enzymes according to this invention may be employed in pharmaceutical formulations as enzyme inhibitors, substrates, pseudosubstrates, inhibitors of DPIV gene expression, binding proteins or antibodies of the target enzyme proteins or as a combination of such different compounds, which reduce DPIV and DPIV-like protein concentration or enzyme activity in mammals. Effectors according to the invention are, for instance, DPIV-inhibitors such as dipeptide derivatives or dipeptide mimetics as alanyl pyrolidide, isoleucyl thiazolidine as well as the pseudosubstrate N-valyl prolyl, O-benzoyl hydroxylamine. Such compounds are known from the literature [DEMUTH, H.-U., Recent developments in the irreversible inhibition of serine and cysteine proteases. J. Enzyme Inhibition 3, 249 (1990)] or may be synthesized according to methods described in the literature.

The method according to the present invention is a new approach to the reduction of elevated circulating glucose concentration in the blood of mammals and to reducing high blood pressure levels.

The present invention relates to the area of dipeptidyl peptidase IV (DPIV) inhibition and, more particularly, to a new use of inhibitors of DPIV and DPIV-like enzyme activity for lowering high blood pressure levels or related disorders in mammals, and pharmaceutical compositions containing said compounds.

In contrast to other proposed methods in the art, the present invention especially provides an orally available therapy with low molecular weight inhibitors of dipeptidyl peptidase IV. The instant invention represents a novel approach for lowering blood pressure levels or related disorders in mammals. It is user friendly, commercially useful and suitable for use in a therapeutic regimen, especially concerning human diseases.

On the basis of these findings, the investigation of the role of DPIV expression and enzymic activity in blood pressure according to the present invention revealed that the oral administration of DPIV inhibitors results in a decrease of blood pressure levels.

The goal of the present invention is the development of dipeptidyl peptidase IV inhibitors and/or ligands, which display a high bioavailability. In another preferred embodiment, the present invention provides DPIV inhibitors, which have an exactly predictable activity time in the target tissue.

Examples for orally available low molecular weight agents are prodrugs of stable and unstable dipeptidyl peptidase IV inhibitors of the general formula A-B-C, wherein A represents an amino acid, B represents the chemical bond between A and C or an amino acid, and C represents an unstable or a stable inhibitor of dipeptidyl peptidase IV respectively. They are described in WO 99/67278 and WO 99/67279 the teachings of which concerning the provision, definition, use and production of the prodrugs are herein incorporated by reference in their entirety. Especially the detailed definitions of A, B and C are herein incorporated by reference.

The present invention relates to a novel method, in which the reduction of activity in the enzyme dipeptidyl peptidase (DPIV or CD26), or of DPIV-like enzyme activity, or where binding of a DPIV specific ligand exerts beneficial effects in the organisms of mammals induced by effectors of the enzyme and leads as a causal consequence to a reduced blood pressure of a mammal. As a consequence mammals having an increased blood pressure will benefit from the treatment with inhibitors of DPIV a DPIV-like enzyme activity.

The method and use according to the present invention comprises preventing increased blood pressure or lowering blood pressure and related disorders in an animal, including humans, by inhibiting DPIV, or related enzyme activities, using an inhibitor or ligand of these enzymes. Oral administration of a DPIV inhibitor may be preferable in most circumstances.

The present invention will now be illustrated with reference to the following examples focusing on the blood pressure and blood glucose lowering action of reduced DPIV-like activity and/or binding.

In one illustrative embodiment, the present invention relates to the use of dipeptide-like compounds and compounds analogous to dipeptide compounds that are formed from an amino acid and a thiazolidine or pyrrolidine group, and salts thereof, referred to hereinafter as dipeptide-like compounds. Preferably the amino acid and the thiazolidine or pyrrolidine group are bonded with an amide bond.

Especially suitable for that purpose according to the invention are dipeptide compounds in which the amino acid is preferably selected from a natural amino acid, such as, for example, leucine, valine, glutamine, glutamic acid, proline, isoleucine, asparagines and aspartic acid.

The dipeptide-like compounds used according to the invention exhibit at a concentration (of dipeptide-compounds) of 10 μM, a reduction in the activity of dipeptidyl peptidase IV or DPIV-analogous enzyme activities of at least 10%, especially of at least 40%. Frequently a reduction in activity of at least 60% or at least 70% is also required. Preferred effectors may also exhibit a reduction in activity of a maximum of 20% or 30%.

Preferred compounds are N-valyl prolyl, O-benzoyl hydroxylamine, alanyl pyrrolidine, isoleucyl thiazolidine like L-allo-isoleucyl thiazolidine, L-threo-isoleucyl pyrrolidine and salts thereof, especially the fumaric salts, and L-allo-isoleucyl pyrrolidine and salts thereof. Especially preferred compounds are glutaminyl pyrrolidine and glutaminyl thiazolidine of formulas 1 and 2: 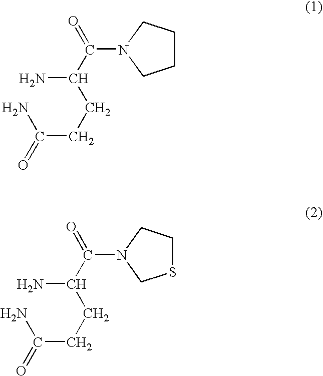
Further preferred compounds are given in Table 1.

The salts of the dipeptide-like compounds can be present in a molar ratio of dipeptide (-analogous) component to salt component of 1:1 or 2:1. Such a salt is, for example, (Ile-Thia)₂ fumaric acid.

TABLE 1
Structures of further preferred dipeptide compounds
Effector

H-Asn-pyrrolidine
H-Asn-thiazolidine
H-Asp-pyrrolidine
H-Asp-thiazolidine
H-Asp(NHOH)-pyrrolidine
H-Asp(NHOH)-thiazolidine
H-Glu-pyrrolidine
H-Glu-thiazolidine
H-Glu(NHOH)-pyrrolidine
H-Glu(NHOH)-thiazolidine
H-His-pyrrolidine
H-His-thiazolidine
H-Pro-pyrrolidine
H-Pro-thiazolidine
H-Ile-azididine
H-Ile-pyrrolidine
H-L-allo-Ile-thiazolidine
H-Val-pyrrolidine
H-Val-thiazolidine

In another preferred embodiment, the present invention provides the use of peptide compounds of formula 3 useful for competitive modulation of dipeptidyl peptidase IV catalysis: 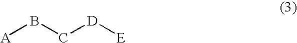
wherein

A, B, C, D and E are independently any amino acid moieties including proteinogenic amino acids, non-proteinogenic amino acids, L-amino acids and D-amino acids and wherein E and/or D may be absent.

Further conditions regarding formula (3):
A is an amino acid except a D-amino acid,
B is an amino acid selected from Pro, Ala, Ser, Gly, Hyp, acetidine-(2)-carboxylic acid and pipecolic acid,
C is any amino acid except Pro, Hyp, acetidine-(2)-carboxylic acid, pipecolic acid and except N-alkylated amino acids, e.g. N-methyl valine and sarcosine,
D is any amino acid or missing, and
E is any amino acid or missing,
   or:
C is any amino acid except Pro, Hyp, acetidine-(2)-carboxylic acid, pipecolic acid, except N-alkylated amino acids, e.g. N-methyl valine and sarcosine, and except a D-amino-acid;
D is any amino acid selected from Pro, Ala, Ser, Gly, Hyp, acetidine-(2)-carboxylic acid and pipecolic acid, and
E is any amino acid except Pro, Hyp, acetidine-(2)-carboxylic acid, pipecolic acid and except N-alkylated amino acids, e.g. N-methyl valine and sarcosine.

Examples of amino acids which can be used in the present invention are L and D-amino acids, N-methyl-amino-acids; allo- and threo-forms of Ile and Thr, which can, e.g. be α-, β- or ω-amino acids, whereof α-amino acids are preferred.

Examples of amino acids throughout the claims and the description are: aspartic acid (Asp), glutamic acid (Glu), arginine (Arg), lysine (Lys), histidine (H is), glycine (Gly), serine (Ser) and cysteine (Cys), threonine (Thr), asparagine (Asn), glutamine (Gln), tyrosine (Tyr), alanine (Ala), proline (Pro), valine (Val), isoleucine (Ile), leucine (Leu), methionine (Met), phenylalanine (Phe), tryptophan (Trp), hydroxyproline (Hyp), beta-alanine (beta-Ala), 2-amino octanoic acid (Aoa), azetidine-(2)-carboxylic acid (Ace), pipecolic acid (Pip), 3-amino propionic, 4-amino butyric and so forth, alpha-aminoisobutyric acid (Aib), sarcosine (Sar), ornithine (Orn), citrulline (Cit), homoarginine (Har), t-butylalanine (t-butyl-Ala), t-butylglycine (t-butyl-Gly), N-methylisoleucine (N-Melle), phenylglycine (Phg), cyclohexylalanine (Cha), norleucine (Nie), cysteic acid (Cya) and methionine sulfoxide (MSO), Acetyl-Lys, modified amino acids such as phosphoryl-serine (Ser(P)), benzyl-serine (Ser(Bzl)) and phosphoryl-tyrosine (Tyr(P)), 2-aminobutyric acid (Abu), aminoethylcysteine (AECys), carboxymethylcysteine (Cmc), dehydroalanine (Dha), dehydroamino-2-butyric acid (Dhb), carboxyglutaminic acid (Gla), homoserine (Hse), hydroxylysine (Hyl), cis-hydroxyproline (cis Hyp), trans-hydroxyproline (transHyp), isovaline (Iva), pyroglutamic acid (Pyr), norvaline (Nva), 2-aminobenzoic acid (2-Abz), 3-aminobenzoic acid (3-Abz), 4-aminobenzoic acid (4-Abz), 4-(aminomethyl)benzoic acid (Amb), 4-(aminomethyl)cyclohexanecarboxylic acid (4-Amc), Penicillamine (Pen), 2-Amino-4-cyanobutyric acid (Cba), cycloalkane-carboxylic acids.

Examples of ω̅-amino acids are e.g.: 5-Ara (aminoraleric acid), 6-Ahx (aminohexanoic acid), 8-Aoc (aminooctanoic acid), 9-Anc (aminovanoic acid), 10-Adc (aminodecanoic acid), 11-Aun (aminoundecanoic acid), 12-Ado (aminododecanoic acid).

Further amino acids are: indanylglycine (Igl), indoline-2-carboxylic acid (Idc), octahydroindole-2-carboxylic acid (Oic), diaminopropionic acid (Dpr), diaminobutyric acid (Dbu), naphtylalanine (1-Nal), (2-Nal), 4-aminophenylalanin (Phe(4-NH₂)), 4-benzoylphenylalanine (Bpa), diphenylalanine (Dip), 4-bromophenylalanine (Phe(4-Br)), 2-chlorophenylalanine (Phe(2-Cl)), 3-chlorophenylalanine (Phe(3-Cl)), 4-chlorophenylalanine (Phe(4-Cl)), 3,4-chlorophenylalanine (Phe (3,4-Cl₂)), 3-fluorophenylalanine (Phe(3-F)), 4-fluorophenylalanine (Phe(4-F)), 3,4-fluorophenylalanine (Phe(3,4-F₂)), pentafluorophenylalanine (Phe(F₅)), 4-guanidinophenylalanine (Phe(4-guanidino)), homophenylalanine (hPhe), 3-jodophenylalanine (Phe(3-J)), 4 jodophenylalanine (Phe(4-J)), 4-methylphenylalanine (Phe(4-Me)), 4-nitrophenylalanine (Phe-4-NO₂)), biphenylalanine (Bip), 4-phosphonomethylphenylalanine (Pmp), cyclohexyglycine (Ghg), 3-pyridinylalanine (3-Pal), 4-pyridinylalanine (4-Pal), 3,4-dehydroproline (A-Pro), 4-ketoproline (Pro(4-keto)), thioproline (Thz), isonipecotic acid (Inp), 1,2,3,4,-tetrahydroisoquinolin-3-carboxylic acid (Tic), propargylglycine (Pra), 6-hydroxynorleucine (NU(6-OH)), homotyrosine (hTyr), 3-jodotyrosine (Tyr(3-J)), 3,5-dijodotyrosine (Tyr(3,5-J₂)), d-methyl-tyrosine (Tyr(Me)), 3-NO₂-tyrosine (Tyr(3-NO₂)), phosphotyrosine (Tyr(PO₃H₂)), alkylglycine, 1-aminoindane-1-carboxy acid, 2-aminoindane-2-carboxy acid (Aic), 4-amino-methylpyrrol-2-carboxylic acid (Py), 4-amino-pyrrolidine-2-carboxylic acid (Abpc), 2-aminotetraline-2-carboxylic acid (Atc), diaminoacetic acid (Gly(NH₂)), diaminobutyric acid (Dab), 1,3-dihydro-2H-isoinole-carboxylic acid (Disc), homocylcohexylalanin (hCha), homophenylalanin (hPhe order Hof), trans-3-phenyl-azetidine-2-carboxylic acid, 4-phenyl-pyrrolidine-2-carboxylic acid, 5-phenyl-pyrrolidine-2-carboxylic acid, 3-pyridylalanine (3-Pya), 4-pyridylalanine (4-Pya), styrylalanine, tetrahydroisoquinoline-1-carboxylic acid (Tiq), 1,2,3,4-tetrahydronorharmane-3-carboxylic acid (Tpi), β-(2-thienyl)-alanine (Tha)

Other amino acid substitutions for those encoded in the genetic code can also be included in peptide compounds within the scope of the invention and can be classified within this general scheme.

Proteinogenic amino acids are defined as natural protein-derived α-amino acids. Non-proteinogenic amino acids are defined as all other amino acids, which are not building blocks of common natural proteins.

The resulting peptides may be synthesized as the free C-terminal acid or as the C-terminal amide form. The free acid peptides or the amides may be varied by side chain modifications. Such side chain modifications include for instance, but not restricted to, homoserine formation, pyroglutamic acid formation, disulphide bond formation, deamidation of asparagine or glutamine residues, methylation, t-butylation, t-butyloxycarbonylation, 4-methylbenzylation, thioanysilation, thiocresylation, bencyloxymethylation, 4-nitrophenylation, bencyloxycarbonylation, 2-nitrobencoylation, 2-nitrosulphenylation, 4-toluenesulphonylation, pentafluorophenylation, diphenylmethylation, 2-chlorobenzyloxycarbonylation, 2,4,5-trichlorophenylation, 2-bromobenzyloxycarbonylation, 9-fluorenylmethyloxycarbonylation, triphenylmethylation, 2,2,5,7,8,-pentamethylchroman-6-sulphonylation, hydroxylation, oxidation of methionine, formylation, acetylation, anisylation, bencylation, bencoylation, trifluoroacetylation, carboxylation of aspartic acid or glutamic acid, phosphorylation, sulphation, cysteinylation, glycolysation with pentoses, deoxyhexoses, hexosamines, hexoses or N-acetylhexosamines, farnesylation, myristolysation, biotinylation, palmitoylation, stearoylation, geranylgeranylation, glutathionylation, 5′-adenosylation, ADP-ribosylation, modification with N-glycolylneuraminic acid, N-acetylneuraminic acid, pyridoxal phosphate, lipoic acid, 4′-phosphopantetheine, or N-hydroxysuccinimide.

In the compounds of formula (3), the amino acid moieties A, B, C, D, and E are respectively attached to the adjacent moiety by amide bonds in a usual manner according to standard nomenclature so that the amino-terminus (N-terminus) of the amino acids (peptide) is drawn on the left and the carboxyl-terminus of the amino acids (peptide) is drawn on the right. (C-terminus)

Until the present invention by Applicants, known peptide substrates of the proline-specific serine protease dipeptidyl peptidase IV in vitro are the tripeptides Diprotin A (Ile-Pro-Ile), Diprotin B (Val-Pro-Leu) and Diprotin C (Val-Pro-Ile). Applicants have unexpectedly discovered that the compounds disclosed herein above and below act as substrates of dipeptidyl peptidase IV in vivo in a mammal and, in pharmacological doses, lower blood pressure and alleviate pathological abnormalities of the metabolism of mammals such as glucosuria, hyperlipidaemia, metabolic acidosis and diabetes mellitus by competitive catalysis.

Particularly preferred compounds of the present invention that are useful as modulators of dipeptidyl peptidase IV and DPIV-like enzymes include those compounds which show Kᵢ-values for DPIV-binding, effectively in DPIV-inhibition in vivo after i.v. and/or p.o. administration to Wistar rats.

Further preferred compounds are peptidylketones of formula 4: 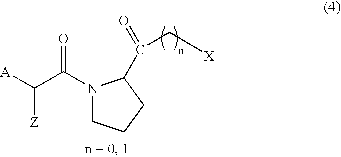
wherein
A is selected from 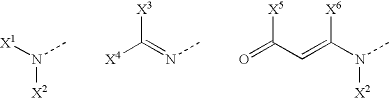
X¹ is H or an acyl or oxycarbonyl group incl. all amino acids and peptide residues,
X² is H, —(CH)ₙ—NH—C₅H₃N—Y with n=2-4 or C₅H₃N—Y (a divalent pyridyl residue) and Y is selected from H, Br, Cl, I, NO₂ or CN,
X³ is H or a phenyl or pyridyl residue, unsubstituted or substituted with one, two or more alkyl, alkoxy, halogen, nitro, cyano or carboxy residues,
X⁴ is H or a phenyl or pyridyl residue, unsubstituted or substituted with one, two or more alkyl, alkoxy, halogen, nitro, cyano or carboxy residues,
X⁵ is H or an alkyl, alkoxy or phenyl residue,
X⁶ is H or an alkyl residue.
   for n=1
X is selected from: H, OR², SR², NR²R³, N⁺R²R³R⁴, wherein:
   R² stands for acyl residues, which are unsubstituted or substituted with one, two or more alkyl, cycloalkyl, aryl or heteroaryl residues, or for all amino acids and peptidic residues, or alkyl residues, which are unsubstituted or substituted with one, two or more alkyl, cycloalkyl, aryl and heteroaryl residues,
   R³ stands for alkyl and acyl functions, wherein R² and R³ may be part of one or more ring structures of saturated and unsaturated carbocyclic or heterocyclic structures,
   R⁴ stands for alkyl residues, wherein R² and R⁴ or R³ and R⁴ may be part of one or more ring structures of saturated and unsaturated carbocyclic or heterocyclic structures,
   for n=0
X is selected from: 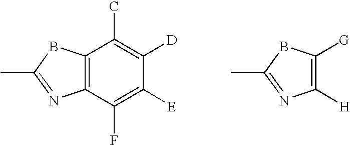
   wherein
B stands for: O, S, NR⁵, wherein R⁵ is H, an alkyliden or acyl,
   C, D, E, F, G, H are independently selected from unsubstituted and substituted alkyl, oxyalkyl, thioalkyl, aminoalkyl, carbonylalkyl, acyl, carbamoyl, aryl and heteroaryl residues; and
      for n=0 and n=1
Z is selected from H, or a branched or single chain alkyl residue from C₁-C₉ or a branched or single chain alkenyl residue from C₂-C₉, a cycloalkyl residue from C₃-C₈, a cycloalkenyl residue from C₅-C₇, an aryl- or heteroaryl residue, or a side chain selected from all side chains of all natural amino acids or derivatives thereof.
   Further, according to the present invention compounds of formulas 5, 6, 7, 8, 9, 10 and 11, including all stereoisomers and pharmaceutical acceptable salts thereof are disclosed and can be used: 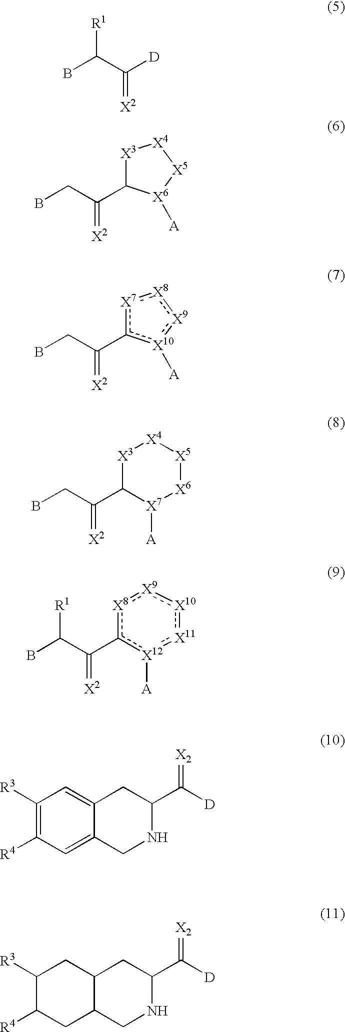
   wherein:
   R¹ is H, a branched or linear C₁-C₉ alkyl residue, a branched or linear C₂-C₉ alkenyl residue, a C₃-C₈ cycloalkyl-, C₅-C₇ cycloalkenyl-, aryl- or heteroaryl residue or a side chain of a natural amino acid or a derivative thereof,
   R³ and R⁴ are selected from H, hydroxy, alkyl, alkoxy, aryloxy, nitro, cyano or halogen,
   A is H or an isoster of a carbonic acid, like a functional group selected from CN, SO₃H, CONHOH, PO₃R⁵R⁶, tetrazole, amide, ester, anhydride, thiazole and imidazole,
   B is selected from: 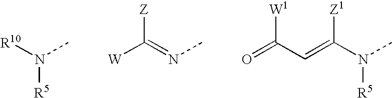
   wherein:
   R⁵ is H, —(CH)ₙ—NH—C₅H₃N—Y with n=2-4 and C₅H₃N—Y (a divalent pyridyl residue) with Y═H, Br, Cl, I, NO₂ or CN
   R¹⁰ is H, an acyl, oxycarbonyl or a amino acid residue,
   W is H or a phenyl or pyridyl residue, unsubstituted or substituted with one, two or more alkyl, alkoxy, halogen, nitro, cyano or carboxy residues,
   W¹ is H, an alkyl, alkoxy or phenyl, residue,
   Z is H or a phenyl or pyridyl residue, unsubstituted or substituted with one, two or more alkyl, alkoxy, halogen, nitro, cyano or carboxy residues,
   Z¹ is H or an alkyl residue,
   D is a cyclic C₄-C₇ alkyl, C₄-C₇ alkenyl residue which can be unsubstituted or substituted with one, two or more alkyl groups or a cyclic 4-7-membered heteroalkyl or a cyclic 4-7-membered heteroalkenyl residue,
   X² is O, NR⁶, N⁺(R⁷)₂, or S,
   X³ to X¹² are independently selected from CH₂, CR⁸R⁹, NR⁶, N⁺(R⁷)₂, O, S, SO and SO₂, including all saturated and unsaturated structures,
   R⁶, R⁷, R⁸, R⁹ are independently selected from H, a branched or linear C1-C₉ alkyl residue, a branched or linear C₂-C₉ alkenyl residue, a C₃-C₈ cycloalkyl residue, a C₅-C₇ cycloalkenyl residue, an aryl or heteroaryl residue,
   with the following provisions:
   Formula 6: X⁶ is CH if A is not H,
   Formula 7: X¹⁰ is C if A is not H,
   Formula 8: X⁷ is CH if A is not H,
   Formula 9: X¹² is C if A is not H.

Throughout the description and the claims the expression “acyl” can denote a C₁₋₂₀ acyl residue, preferably a C₁₋₈ acyl residue and especially preferred a C₁₋₄ acyl residue, “cycloalkyl” can denote a C₃₋₁₂ cycloalkyl residue, preferably a C₄, C₅ or C₆ cycloalkyl residue, “carbocyclic” can denote a C₃₋₁₂ carbocyclic residue, preferably a C₄, C₅ or C₆ carbocyclic residue. “Heteroaryl” is defined as an aryl residue, wherein 1 to 4, preferably 1, 2 or 3 ring atoms are replaced by heteroatoms like N, S or O. “Heterocyclic” is defined as a cycloalkyl residue, wherein 1, 2 or 3 ring atoms are replaced by heteroatoms like N, S or O. “Peptides” are selected from dipeptides to decapeptides, preferred are dipeptides, tripeptides, tetrapeptides and pentapeptides. The amino acids for the formation of the “peptides” can be selected from the those listed above.

Because of the wide distribution of the protein in the body and the wide variety of mechanisms involving DPIV, DPIV-activity and DPIV-related proteins, systemic therapy (enteral or parenteral administration) with DPIV-inhibitors can result in a series of undesirable side-effects.

The problem to be solved was moreover, to provide compounds that can be used for targeted influencing of locally limited pathophysiological and physiological processes. The problem of the invention especially consists in obtaining locally limited inhibition of DPIV or DPIV-analogous activity for the purpose of targeted intervention in the regulation of the activity of locally active substrates.

This problem is solved according to the invention by compounds of the general formula (12) 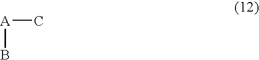
wherein
A is an amino acid having at least one functional group in the side chain,
B is a chemical compound covalently bound to at least one functional group of the side chain of A,
C is a thiazolidine, pyrrolidine, cyanopyrrolidine, hydroxyproline, dehydroproline or piperidine group amide-bonded to A.
The compounds can, e.g., be used for reducing blood pressure by acting on the DPIV or DPIV-like enzymes in the endothelium of blood vessels.

In accordance with a preferred embodiment of the invention, pharmaceutical compositions are used comprising at least one compound of the general formula (12) and at least one customary adjuvant appropriate for the site of action.

Preferably A is an α-amino acid, especially a natural 1-amino acid having one, two or more functional groups in the side chain, preferably threonine, tyrosine, serine, arginine, lysine, aspartic acid, glutamic acid or cysteine.

Preferably B is an oligopeptide having a chain length of up to 20 amino acids, a polyethylene glycol having a molar mass of up to 20 000 g/mol, an optionally substituted organic amine, amide, alcohol, acid or aromatic compound having from 8 to 50 C atoms.

Throughout the description and the claims the expression “alkyl” can denote a C₁₋₅₀ alkyl group, preferably a C₆₋₃₀ alkyl group, especially a C₈₋₁₂ alkyl group; for example, an alkyl group may be a methyl, ethyl, propyl, isopropyl or butyl group. The expression “alk”, for example in the expression “alkoxy”, and the expression “alkan”, for example in the expression “alkanoyl”, are defined as for “alkyl”; aromatic compounds are preferably substituted or optionally unsubstituted phenyl, benzyl, naphthyl, biphenyl or anthracene groups, which preferably have at least 8 C atoms; the expression “alkenyl” can denote a C₂₋₁₀ alkenyl group, preferably a C₂₋₆ alkenyl group, which has the double bond(s) at any desired location and may be substituted or unsubstituted; the expression “alkynyl” can denote a C₂₋₁₀ alkynyl group, preferably a C₂₋₆ alkynyl group, which has the triple bond(s) at any desired location and may be substituted or unsubstituted; the expression “substituted” or substituent can denote any desired substitution by one or more, preferably one or two, alkyl, alkenyl, alkynyl, mono- or multi-valent acyl, alkanoyl, alkoxyalkanoyl or alkoxyalkyl groups; the afore-mentioned substituents may in turn have one or more (but preferably zero) alkyl, alkenyl, alkynyl, mono- or multi-valent acyl, alkanoyl, alkoxyalkanoyl or alkoxyalkyl groups as side groups; organic amines, amides, alcohols or acids, each having from 8 to 50 C atoms, preferably from 10 to 20 C atoms, can have the formulae (alkyl)₂N— or alkyl-NH—, —CO—N(alkyl)₂ or —CO—NH(alkyl), -alkyl-OH or -alkyl-COOH.

Despite an extended side chain function, the compounds of formula (12) can still bind to the active centre of the enzyme dipeptidyl peptidase IV and analogous enzymes but are no longer actively transported by the peptide transporter PepT1. The resulting reduced or greatly restricted transportability of the compounds according to the invention leads to local or site directed inhibition of DPIV and DPIV-like enzyme activity.

The compounds of formula (12) or the other compounds and prodrugs used in accordance with the invention can be present or used, respectively, in the form of racemates or in the form of enantiomerically pure compounds, preferably in the L-threo or L-allo form with respect to part A of formula (12).

By extending/expanding the side chain modifications, for example beyond a number of seven carbon atoms, it is accordingly possible to obtain a dramatic reduction in transportability (see Example 12). The Examples in Table 12.1 clearly show that, with increasing spatial size of the side chains, there is a reduction in the transportability of the substances. By spatially and sterically expanding the side chains, for example beyond the atom group size of a monosubstituted phenyl radical, hydroxylamine radical or amino acid residue, it is possible according to the invention to modify or suppress the transportability of the target substances.

According to the present invention, the compounds of formula (12) inhibit DPIV or DPIV-like enzyme activity in the body of a mammal in a site specific manner. It is accordingly possible to influence local physiological and pathophysiological conditions (inflammation, psoriasis, arthritis, autoimmune diseases, allergies, cancer, metastasis, blood pressure in the endothelium of blood vessels) effectively and with dramatically reduced side-effects.

Preferred compounds of formula (12) are compounds, wherein the oligopeptides have chain lengths of from 3 to 15, especially from 4 to 10, amino acids, and/or the polyethylene glycols have molar masses of at least 250 g/mol, preferably of at least 1500 g/mol and up to 15 000 g/mol, and/or the optionally substituted organic amines, amides, alcohols, acids or aromatic compounds have at least 12 C atoms and preferably up to 30 C atoms.

The compounds of the present invention can be converted into and used as acid addition salts, especially pharmaceutically acceptable acid addition salts. The pharmaceutically acceptable salt generally takes a form in which an amino acids basic side chain is protonated with an inorganic or organic acid. Representative organic or inorganic acids include hydrochloric, hydrobromic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benzenesulfonic, oxalic, pamoic, 2-naphthalenesulfonic, p-toulenesulfonic, cyclohexanesulfamic, salicylic, saccharinic or trifluoroacetic acid. All pharmaceutically acceptable acid addition salt forms of the compounds of formulas (1) to (12) are intended to be embraced by the scope of this invention.

In view of the close relationship between the free compounds and the compounds in the form of their salts, whenever a compound is referred to in this context, a corresponding salt is also intended, provided such is possible or appropriate under the circumstances.

The present invention further includes within its scope prodrugs of the compounds of this invention. In general, such prodrugs will be functional derivatives of the compounds which are readily convertible in vivo into the desired therapeutically active compound. Thus, in these cases, the use of the present invention shall encompass the treatment of the various disorders described with prodrug versions of one or more of the claimed compounds, which convert to the above specified compound in vivo after administration to the subject. Conventional procedures for the selection and preparation of suitable prodrug derivatives are described, for example, in “Design of Prodrugs”, ed. H. Bundgaard, Elsevier, 1985 and the patent applications DE 198 28 113 and DE 198 28 114, which are fully incorporated herein by reference.

Where the compounds or prodrugs according to this invention have at least one chiral center, they may accordingly exist as enantiomers. Where the compounds or prodrugs possess two or more chiral centers, they may additionally exist as diastereomers. It is to be understood that all such isomers and mixtures thereof are encompassed within the scope of the present invention. Furthermore, some of the crystalline forms of the compounds or prodrugs may exist as polymorphs and as such are intended to be included in the present invention. In addition, some of the compounds may form solvates with water (i.e. hydrates) or common organic solvents, and such solvates are also intended to be encompassed within the scope of this invention.

The compounds, including their salts, can also be obtained in the form of their hydrates, or include other solvents used for their crystallization.

As indicated above, the compounds and prodrugs of the present invention, and their corresponding pharmaceutically acceptable acid addition salt forms, are useful in inhibiting DPIV and DPIV-like enzyme activity. The ability of the compounds and prodrugs of the present invention, and their corresponding pharmaceutically acceptable acid addition salt forms to inhibit DPIV and DPIV-like enzyme activity may be demonstrated employing the DPIV activity assay for determination of the Kᵢ-values and the IC₅₀-values in vitro, as described in examples 7 and 8.

The ability of the compounds of the present invention, and their corresponding pharmaceutically acceptable acid addition salt forms to inhibit DPIV in vivo may be demonstrated by oral or intravasal administration to Wistar rats, as described in example 11. The compounds of the present invention inhibit DPIV activity in vivo after both, oral and intravasal administration to Wistar rats.

DPIV is present in a wide variety of mammalian organs and tissues e.g. the intestinal brush-border (Gutschmidt S. et al., “In situ”-measurements of protein contents in the brush border region along rat jejunal villi and their correlations with four enzyme activities. Histochemistry 1981, 72 (3), 467-79), exocrine epithelia, hepatocytes, renal tubuli, endothelia, myofibroblasts (Feller A. C. et al., A monoclonal antibody detecting dipeptidylpeptidase IV in human tissue. Virchows Arch. A. Pathol. Anat. Histopathol. 1986; 409 (2):263-73), nerve cells, lateral membranes of certain surface epithelia, e.g. Fallopian tube, uterus and vesicular gland, in the luminal cytoplasm of e.g., vesicular gland epithelium, and in mucous cells of Brunner's gland (Hartel S. et al., Dipeptidyl peptidase (DPP) IV in rat organs. Comparison of immunohistochemistry and activity histochemistry. Histochemistry 1988; 89 (2): 151-61), reproductive organs, e.g. cauda epididymis and ampulla, seminal vesicles and their secretions (Agrawal & Vanha-Perttula, Dipeptidyl peptidases in bovine reproductive organs and secretions. Int. J. Androl. 1986, 9 (6): 435-52). In human serum, two molecular forms of dipeptidyl peptidase are present (Krepela E. et al., Demonstration of two molecular forms of dipeptidyl peptidase IV in normal human serum. Physiol. Bohemoslov. 1983, 32 (6): 486-96). The serum high molecular weight form of DPIV is expressed on the surface of activated T cells (Duke-Cohan J. S. et al., Serum high molecular weight dipeptidyl peptidase IV (CD26) is similar to a novel antigen DPPT-L released from activated T cells. J. Immunol. 1996, 156 (5): 1714-21).

The compounds and prodrugs of the present invention, and their corresponding pharmaceutically acceptable acid addition salt forms are able to inhibit DPIV in vivo. In one embodiment of the present invention, all molecular forms, homologues and epitopes of DPIV from all mammalian tissues and organs, also of those, which are undiscovered yet, are intended to be embraced by the scope of this invention.

Among the rare group of proline-specific proteases, DPIV was originally believed to be the only membrane-bound enzyme specific for proline as the penultimate residue at the amino-terminus of the polypeptide chain. However, other molecules, even structurally non-homologous with the DPIV but bearing corresponding enzyme activity, have been identified recently. DPIV-like enzymes, which are identified so far, are e.g. fibroblast activation protein α, dipeptidyl peptidase IV β, dipeptidyl aminopeptidase-like protein, N-acetylated α-linked acidic dipeptidase, quiescent cell proline dipeptidase, dipeptidyl peptidase II, attractin and dipeptidyl peptidase IV related protein (DPP 8), and are described in the review article by Sedo & Malik (Sedo & Malik, Dipeptidyl peptidase IV-like molecules: homologous proteins or homologous activities? Biochimica et Biophysica Acta 2001, 36506: 1-10). Further DPIV like enzymes are disclosed in WO 01/19866, WO 02/04610 and WO 02/34900. WO 01/19866 discloses novel human dipeptidyl aminopeptidase (DPP8) with structural and functional similarities to DPIV and fibroblast activation protein (FAP). The dipeptidyl peptidase IV-like enzyme of WO 02/04610 is well known in the art. In the Gene Bank data base, this enzyme is registered as KIAA1492. In another preferred embodiment of the present invention, all molecular forms, homologues and epitopes of proteins comprising DPIV-like enzyme activity, from all mammalian tissues and organs, also of those, which are undiscovered yet, are intended to be embraced by the scope of this invention.

The ability of the compounds and prodrugs of the present invention, and their corresponding pharmaceutically acceptable acid addition salt forms to inhibit DPIV-like enzymes may be demonstrated employing an enzyme activity assay for determination of the Kᵢ-values in vitro as described in example 9. The Kᵢ-values of the compounds of the present invention against porcine dipeptidyl peptidase II were exemplary determined as Kᵢ=8.52*10⁻⁵ M±6.33*10⁻⁶ M for glutaminyl pyrrolidine and Kᵢ=1.07*10⁻⁵ M±3.81*10⁻⁷ M for glutaminyl thiazolidine.

In another embodiment, the compounds and prodrugs of the present invention, and their corresponding pharmaceutically acceptable acid addition salt forms have only low, if no inhibitory activity against non-DPIV and non-DPIV-like proline specific enzymes. As described in example 10, with glutaminyl thiazolidine and glutaminyl pyrrolidine exemplarily, no inhibition of dipeptidyl peptidase I and prolyl oligopeptidase was found. Against prolidase, both compounds showed a marked lower efficacy compared to DPIV. The IC 50-values against prolidase were determined as IC 50>3 mM for glutaminyl thiazolidine and as IC 50=3.4*10⁻⁴M±5.63*10⁻⁵ for glutaminyl pyrrolidine.

The present invention provides a method of preventing or treating a condition mediated by modulation of the DPIV or DPIV-like enzyme activity in a subject in need thereof which comprises administering any of the compounds of the present invention or pharmaceutical compositions thereof in a quantity and dosing regimen therapeutically effective to treat the condition. Additionally, the present invention includes the use of the compounds and prodrugs of this invention, and their corresponding pharmaceutically acceptable acid addition salt forms, for the preparation of a medicament for the prevention or treatment of a condition mediated by modulation of the DPIV activity in a subject. The compound may be administered to a patient by any conventional route of administration, including, but not limited to, intravenous, oral, subcutaneous, intramuscular, intradermal, parenteral and combinations thereof.

In a further illustrative embodiment, the present invention provides formulations for the compounds of formulas 1 to 12, and their corresponding pharmaceutically acceptable prodrugs and acid addition salt forms, in pharmaceutical compositions.

The term “subject” as used herein, refers to an animal, preferably a mammal, most preferably a human, who has been the object of treatment, observation or experiment.

The term “therapeutically effective amount” as used herein, means that amount of active compound or pharmaceutical agent that elicits the biological or medicinal response in a tissue system, animal or human, being sought by a researcher, veterinarian, medical doctor or other clinician, which includes alleviation of the symptoms of the disease or disorder being treated.

As used herein, the term “composition” is intended to encompass a product comprising the claimed compounds in the therapeutically effective amounts, as well as any product which results, directly or indirectly, from combinations of the claimed compounds.

To prepare the pharmaceutical compositions used in this invention, one or more compounds of formulas 1 to 12, or their corresponding pharmaceutically acceptable prodrugs or acid addition salt forms, as the active ingredient, is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives may advantageously include water, glycols, oils, alcohols, flavoring agents, preservatives, coloring agents and the like; for solid oral preparations such as, for example, powders, capsules, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are employed. If desired, tablets may be sugar coated or enteric coated by standard techniques. For parenterals the carrier will usually comprise sterile water, through other ingredients, for example, for purposes such as aiding solubility or for preservation, may be included.

Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per dosage unit, e.g., tablet, capsule, powder, injection, suppository, teaspoonful and the like, of from about 0.01 mg to about 1000 mg (preferably about 5 to about 500 mg) and may be given at a dosage of from about 0.1 to about 300 mg/kg bodyweight per day (preferably 1 to 50 mg/kg per day). The dosages, however, may be varied depending upon the requirement of the patients, the severity of the condition being treated and the compound being employed. The use of either daily administration or post-periodic dosing may be employed. Typically the dosage will be regulated by the physician based on the characteristics of the patient, his/her condition and the therapeutic effect desired.

Preferably these compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is ideally mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is ideally dispersed evenly throughout the composition so that the composition may be readily subdivided into equally effective dosage forms such as tablets, pills and capsules. This solid preformulation composition may then be subdivided into unit dosage forms of the type described above containing from about 0.01 to about 1000 mg, preferably from about 5 to about 500 mg of the active ingredient of the present invention.

The tablets or pills of the novel composition can be advantageously coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids with such materials as shellac, cetyl alcohol and cellulose acetate.

This liquid forms in which the novel compositions of the present invention may be advantageously incorporated for administration orally or by injection include, aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinylpyrrolidone or gelatin.

Where the processes for the preparation of the compounds according to the invention give rise to a mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their components enantiomers by standard techniques, such as the formation of diastereomeric pairs by salt formation with an optically active acid, such as (−)-di-p-toluoyl-d-tartaric acid and/or (+)-di-p-toluoyl-l-tartaric acid followed by fractional crystallization and regeneration of the free base. The compounds may also resolved by formation of diastereomeric esters or amides, followed by chromatographic separation and removal of is the chiral auxiliary. Alternatively, the compounds may be resolved using a chiral HPLC column.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991, fully incorporated herein by reference. The protecting groups may be removed at a convenient subsequent stage using methods known from the art.

The method of treating conditions modulated by dipeptidyl peptidase IV and DPIV-like enzymes described in the present invention may also be carried out using a pharmaceutical composition comprising one or more of the compounds as defined herein and a pharmaceutically acceptable carrier. The pharmaceutical composition may contain from about 0.01 mg to 1000 mg, preferably about 5 to about 500 mg, of the compound(s), and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixirs, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds of the present invention can be administered in intranasal form via topical use of suitable intranasal vehicles, or via transdermal skin patches well known to those of ordinary skill in that art. To be administered in the form of transdermal delivery system, the dosage administration will, of course, be continuous rather than intermittent throughout the dosage regimen and dosage strength will need to be accordingly modified to obtain the desired therapeutic effects.

More preferably, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or betalactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and other compounds known within the art.

The liquid forms are suitable in flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

The compound of the present invention can also be administered in the form of liposome delivery systems, such as small unilamellar vesicles, large unilamellar vesicles, and multilamellar vesicles. Liposomes can be formed from a variety of phospholipids, such as cholesterol, stearylamine or phosphatidylcholines using processes well described in the art.

Compounds of the present invention may also be coupled with soluble polymers as targetable drug carriers. Such polymers can include polyvinylpyrrolidone, pyran copolymer, polyhydroxypropylmethacrylamidephenol, polyhydroxyethylaspartamide-phenol, or polyethyl eneoxidepolyllysine substituted with palmitoyl residue. Furthermore, compounds of the present invention may be coupled to a class of biodegradable polymers useful in achieving controlled release of a drug, for example, polyacetic acid, polyepsilon caprolactone, polyhydroxy butyeric acid, polyorthoesters, polyacetals, polydihydropyrans, polycyanoacrylates and cross-linked or amphipathic block copolymers of hydrogels.

Compounds of this invention may be administered in any of the foregoing compositions and according to dosage regimens established in the art whenever treatment of the addressed disorders is required.

The daily dosage of the products may be varied over a wide range from 0.01 to 1.000 mg per adult human per day. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250, 500 and 1000 milligrams of the active ingredient for the symptomatic adjustment of the dosage to the patient to be treated. An effective amount of the drug is ordinarily supplied at a dosage level of from about 0.1 mg/kg to about 300 mg/kg of body weight per day. Preferably, the range is from about 1 to about 50 mg/kg of body weight per day. The compounds may be administered on a regimen of 1 to 4 times per day.

Optimal dosages to be administered may be readily determined by those skilled in the art, and will vary with the particular compound used, the mode of administration, the strength of the preparation, bioavailability due to the mode of administration, and the advancement of disease condition. In addition, factors associated with the particular patient being treated, including patient age, weight, diet and time of administration, should generally be considered in adjusting dosages.

The compounds or compositions of the present invention may be taken before a meal e.g. 1 hour, 30, 15 or 5 min before eating or drinking, while taking a meal or after a meal.

When taken while eating, the compounds or compositions of the present invention can be mixed into the meal or taken in a separate dosage form as described above.

### EXAMPLES

### Example 1

### Synthesis of Dipeptide-Like Compounds

1.1 General Synthesis of Isoleucyl Thiazolidine Salt

The Boc-protected amino acid BOC-Ile-OH is placed in ethyl acetate and the batch is cooled to about −5° C. N-Methylmorpholine is added dropwise, pivalic acid chloride (on a laboratory scale) or neohexanoyl chloride (on a pilot-plant scale) is added dropwise at constant temperature. The reaction is stirred for a few minutes for activation. N-Methylmorpholine (laboratory scale) and thiazolidine hydrochloride (laboratory scale) are added dropwise in succession, thiazolidine (pilot-plant scale) is added. Working-up in the laboratory is effected in conventional manner using salt solutions, on a pilot-plant scale the batch is purified with NaOH and CH₃COOH solutions.

The removal of the BOC protecting group is carried out using HCl/dioxane (laboratory scale) or H₂SO₄ (pilot-plant scale). In the laboratory the hydrochloride is crystallised from EtOH/ether.

On a pilot-plant scale the free amine is prepared by the addition of NaOH/NH₃. Fumaric acid is dissolved in hot ethanol, the free amine is added dropwise, and (Ile-Thia)² fumarate (M=520.71 gmol⁻¹) precipitates. The analysis of isomers and enantiomers is carried out by electrophoresis.

1.2 Synthesis of Glutaminyl Pyrrolidine Free Base

Acylation:

N-Benzyl-oxycarbonylglutamine (2.02 g, 7.21 mmol) was dissolved in 35 ml THF and brought to −15° C. Into that mixture CAIBE (isobutylchloroformate) (0.937 ml, 7.21 mmol) and 4-methylmorpholine (0.795 ml, 7.21 mmol) where added and the solution was stirred for 15 min. The formation of the mixed anhydride was checked by TLC (eluent: CHCl₃/MeOH: 9/1). After warming to −10° C. pyrrolidine (0.596 ml, 7.21 mmol) was added. The mixture was brought to room temperature and stirred overnight.

Workup:

The sediment formed was filtered off and the solvent was evaporated. The resulting oil was taken up in ethylacetate (20 ml) and washed with a saturated solution of sodiumhydrogensulfate followed by a saturated solution of sodiumbicarbonate, water and brine. The organic layer was separated, dried and evaporated. The resulting product was checked for purity by TLC (eluent: CHCl₃/MeOH: 9/1)

Yield: 1.18 g, waxy solid

Cleavage:

1.18 g of the resulting solid Z-protected compound was dissolved in 40 ml absolute ethanol. Into the solution ca. 20 mg Pd on charcoal (10%, FLUKA) was added and the suspension was shaken under a hydrogen atmosphere for 3 h. The progress of the reaction was monitored by TLC (eluent: CHCl₃/MeOH: 9/1). After completion of the reaction the was removed to provide the free base.

Yield: 99%

The purity was checked by means of TLC: n-butanole/AcOH/water/ethylacetate: 1/1/1/1, R_{f}=0.4. The identity of the reaction product was checked by NMR analysis.

1.3 Synthesis of Glutaminyl Thiazolidine Hydrochloride

Acylation:

N-t-Butyl-oxycarbonylglutamine (2.0 g, 8.12 mmol) was dissolved in 5 ml THF and brought to −15° C. Into that mixture CAIBE (isobutylchloroformate) (1.06 ml, 8.12 mmol) and 4-methylmorpholine (0.895 ml, 8.12 mmol) where added and the solution was stirred for 15 min. The formation of the mixed anhydride was checked by TLC (eluent: CHCl₃/MeOH: 9/1). After warming to −10° C. another equivalent 4-methylmorpholine (0.895 ml, 8.12 mmol) and thiazolidinehydrochloride (1.02 g, 8.12 mmol was added. The mixture was brought to room temperature and stirred overnight.

Workup:

The sediment formed was filtered off and the solvent was evaporated. The resulting oil was taken up in chloroform (20 ml) and washed with a saturated solution of sodiumhydrogensulfate followed by a saturated solution of sodiumbicarbonate, water and brine. The organic layer was separated, dried and evaporated. The resulting product was checked for purity by TLC (eluent: CHCl₃/MeOH: 9/1)

Yield: 1.64 g, solid

Cleavage:

640 mg of the resulting solid Boc-protected compound was dissolved in 3.1 ml ice cold HCl in dioxane (12.98 M, 20 equivalents) and left on ice. The progress of the reaction was monitored by TLC (eluent: CHCl₃/MeOH: 9/1). After completion of the reaction the solvent was removed and the resulting oil was taken up in methanole and evaporated again. After that the resulting oil was dried over phosphorous-V-oxide and triturated two times with diethylether. The purity was checked by HPLC.

Yield: 0.265 g

The purity was checked by HPLC. The identity of the reaction product was checked by NMR analysis.

1.4 Synthesis of Glutaminyl Pyrrolidine Hydrochloride

Acylation:

N-t-Butyl-oxycarbonylglutamine (3.0 g, 12.18 mmol) was dissolved in 7 ml THF and brought to −15° C. Into that mixture CAIBE (isobutylchloroformate) (1.6 ml, 12.18 mmol) and 4-methylmorpholine (1.3 ml, 12.18 mmol) where added and the solution was stirred for 15 min. The formation of the mixed anhydride was checked by TLC (eluent: CHCl₃/MeOH: 9/1). After warming to −10° C. 1 equivalent of pyrrolidine (1.0 ml, 12.18 mmol) was added. The mixture was brought to room temperature and stirred overnight.

Workup:

The sediment formed was filtered off and the solvent was evaporated. The resulting oil was taken up in chloroform (20 ml) and washed with a saturated solution of sodiumhydrogensulfate followed by a saturated solution of sodiumbicarbonate, water and brine. The organic layer was separated, dried and evaporated. The resulting product was checked for purity by TLC (eluent: CHCl₃/MeOH: 9/1)

Yield: 2.7 g solid

Cleavage:

2.7 g of the resulting solid was dissolved in 13.0 ml ice cold HCl in dioxane (12.98 M, 20 equivalents) and left on ice. The progress of the reaction was monitored by TLC (eluent: CHCl₃/MeOH: 9/1). After completion of the reaction the solvent was removed and the resulting oil was taken up in methanole and evaporated again. After that the resulting oil was dried over phosphorous-V-oxide and triturated two times with diethylether.

Yield: 980 mg

The purity was checked by HPLC. The identity of the reaction product was checked by NMR analysis.

### Example 2

### Chemical Characterization of Selected Dipeptide Compounds

2.1 Melting Point Determination

Melting points were determined on a Kofler heating platform microscope from Leica Aktiengesellschaft, the values are not corrected, or on a DSC apparatus (Heumann-Pharma).

2.2 Optical Rotation

The rotation values were recorded at different wavelengths on a “Polarimeter 341” or higher, from the Perkin-Elmer company.

2.3 Measurement Conditions for the Mass Spectroscopy

The mass spectra were recorded by means of electrospray ionisation (ESI) on an “API 165” or API 365” from the PE Sciex company. The operation is carried out using an approximate concentration of c=10 μg/ml, the substance is taken up in MeOH/H₂O 50:50, 0.1% HCO₂H, the infusion is effected using a spray pump (20 μl/min). The measurement were made in positive mode [M+H]⁺, the ESI voltage is U=5600V.

2.4. Results

2.4.1 Tests on isoleucyl thiazolidine fumarate (isomer)

| Substance | Mp (° C.) | CE (min) | MS | [α]H₂O |
|---|---|---|---|---|
| L-threo-IT*F | 150^{DSC} | 160 | 203 | −10.7 |
| | | | | (405 nm) |
| D-threo-IT*F | 147 | 158 | 203 | not |
| | | | | determined |
| L-allo-IT*F | 145-6 | 154 | 203 | −4.58 |
| | | | | (380 nm) |
| D-allo-IT*F | 144-6 | 150 | 203 | 4.5 |
| | | | | (380 nm) |

IT*F = isoleucyl thiazolidine fumarate
The NMR and HPLC data confirm the identity of the substance in question.

2.4.2 Tests on other isoleucyl thiazolidine salts

| IT*salt | M (gmol⁻¹) | MP (° C.) |
|---|---|---|
| succinate | 522.73 | 116 |
| tartrate | 352.41 | 122 |
| fumarate | 520.71 | 156 |
| hydrochloride | 238.77 | 169 |
| phosphate | 300.32 | 105 |

### Example 3

### Synthesis of Xaa-Pro-Yaa Tripeptides

All syntheses were carried out on a peptide synthesizer SP 650 (Labortec AG) applying Fmoc/tBu-strategy. Protected amino acids were purchased from Novabiochem or Bachem. trifluoro acetic acid (TFA) was purchased from Merck, triisopropyl silane (TIS) was purchased from Fluka.

Pre-loaded Fmoc-Yaa-Wang resin (2.8 g/substitution level 0.57 mmol/g) was deprotected using 20% piperidine/N,N-dimethylformamide (DMF). After washing with DMF a solution of 2 eq (1.1 g) of Fmoc-Pro-OH were solved in DMF (12 ml solvent per gram resin). 2 eq (1.04 g) of 2-(1H-Benzotriazole 1-yl)-1,1,3,3-tetramethyluronium tetrafluoroborate (TBTU) and 4 eq (1.11 ml) of N,N-diisopropylethylamine (DIEA) were added and placed in the reaction vessel. The mixture was shaken at room temperature for 20 minutes. Then the coupling cycle was repeated. After subsequent washing with DMF, dichlormethane, isopropanol and diethyl ether the resulting Fmoc-Pro-Ile-Wang resin was dried and then divided into 6 parts before coupling the last amino acid derivative.

Fmoc protecting group was removed as described above. After that 0.54 mmol of the Boc-amino acid, 0.54 mmol TBTU and 0.108 mmol DIEA in DMF were shaken for 20 min. The coupling cycle was repeated. Finally the peptide resin washed and dried described above.

The peptide was cleaved from the resin using a mixture of trifluoroacetic acid (TFA) for 2.5 h, containing the following scavengers: TFA/H₂O/triisipropylsilane (TIS)=9.5/0.25/0.25

The yields of crude peptides were 80-90% on the average. The crude peptide was purified by HPLC on a Nucleosil C18 column (7 μm, 250*21.20 mm, 100 A) using a linear gradient of 0.1% TFA/H₂O with increasing concentration of 0.1% TFA/acetonitrile (from 5% to 65% in 40 min) at 6 ml/min.

The pure peptide was obtained by lyophilization, identified by Electrospray mass spectrometry and HPLC analysis.

3.1 Results - Identification of Xaa-Pro-Yaa tripeptides after
chemical synthesis

| | | Mass (exp.)¹ | |
|---|---|---|---|
| Peptide | Mass (calc.) | [M + H⁺] | HPLC k′² |
| Abu-Pro-Ile | 313.4 | 314.0 | 5.7 |
| Cha-Pro-Ile | 381.52 | 382.0 | 10.4 |
| Nva-Pro-Ile | 327.43 | 328.2 | 6.82 |
| Phg-Pro-Ile | 361.44 | 362.2 | 7.9 |
| Nle-Pro-Ile | 341.45 | 342.2 | 8.09 |
| Pip-Pro-Ile | 338.56 | 340.0 | 6.5 |
| Thr-Pro-Ile | 329.4 | 330.0 | 5.12 |
| Trp-Pro-Ile | 414.51 | 415.2 | 9.85 |
| Phe-Pro-Ile | 375.47 | 376.2 | 8.96 |
| Ser-Pro-Ile | 315.37 | 316.3 | 5.24 |
| Ser(P)-Pro-Ile | 395.37 | 396.0 | 3.35 |
| Tyr(P)-Pro-Ile | 471.47 | 472.3 | 5.14 |
| Val-Pro-Val | 313.4 | 314.0 | 5.07 |
| Ile-Pro-Val | 327.43 | 328.5 | 6.41 |
| Ile-Pro-allo-Ile | 341.4 | 342.0 | 7.72 |
| Val-Pro-allo-Ile | 327.4 | 328.5 | 6.51 |
| Tyr-Pro-allo-Ile | 391.5 | 392.0 | 7.02 |
| 2-Amino octanoic acid- | 369.5 | 370.2 | 10.63 |
| Pro-Ile | | | |
| Ser(Bzl)-Pro-Ile | 405.49 | 406.0 | 9.87 |
| Orn-Pro-Ile | 342.42 | 343.1 | 3.73 |
| Tic-Pro-Ile | 387.46 | 388.0 | 8.57 |
| Aze-Pro-Ile | 311.4 | 312.4 | 5.29 |
| Aib-Pro-Ile | 313.4 | 314.0 | 5.25 |
| t-butyl-Gly-Pro-Ile | 341.47 | 342.1 | 7.16 |
| Ile-Hyp-Ile | 356.45 | 358.2 | 6.57 |
| t-butyl-Gly-Pro-Val | 327.4 | 328.4 | 6.32 |
| t-butyl-Gly-Pro-Gly | 285.4 | 286.3 | 3.74 |
| t-butyl-Gly-Pro-Ile-amide | 340.47 | 341.3 | 7.8 |
| t-butyl-Gly-Pro-D-Val | 327.4 | 328.6 | 7.27 |
| t-butyl-Gly-Pro-t-butyl-Gly | 341.24 | 342.5 | 9.09 |
| Ile-Pro-t-butyl-Gly | 341.47 | 342.36 | 6.93 |
| Val-Pro-t-butyl-Gly | 327.4 | 328.15 | 5.98 |

¹[M + H⁺] were determined by Electrospray mass spectrometry in positive ionization mode.
²RP-HPLC conditions:
column: LiChrospher 100 RP 18 (5 μm), 125 × 4 mm
detection (UV): 214 nm
gradient system: acetonitrile (ACN)/H₂O (0.1% TFA) from 5% ACN to 50% in 15 min,
flow: 1 ml/min
k′ = (tᵣ − t₀)/t₀
t₀ = 1.16 min

t-butyl-Gly is defined as: 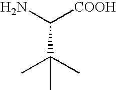
Ser(Bzl) and Ser(P) are defined as benzylserine and phosphorylserine, respectively. Tyr(P) is defined as phosphoryltyrosine.

### Example 4

### Synthesis of Peptidylketones

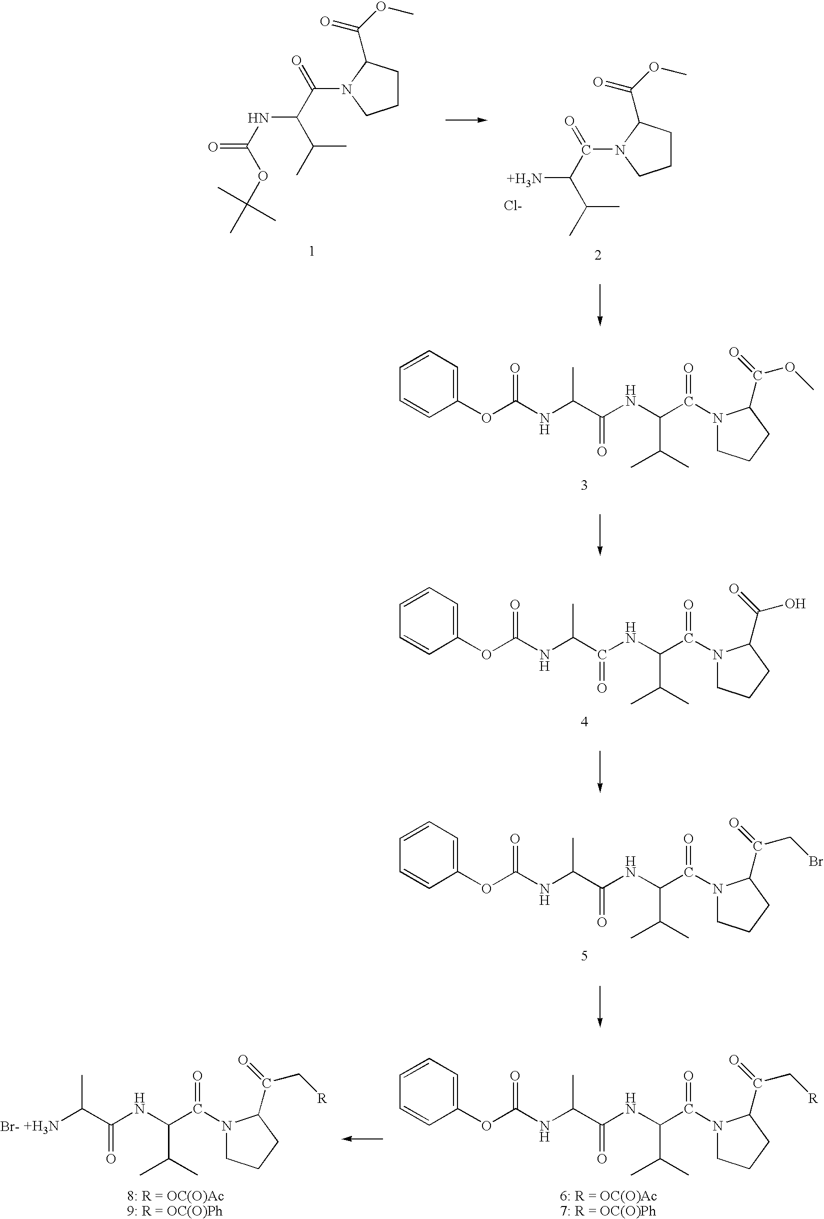 H-Val-Pro-OMe*HCl 2

Boc-Val-OH (3.00 g, 13.8 mmol) was dissolved in 10 ml of dry THF and cooled down to −15° C. To the mixture CAIBE (1.80 ml, 13.8 mmol) and NMM (1.52 ml, 13.8 mmol) where added and the solution was stirred until the formation of the mixed anhydride was complete. Then the mixture was brought to −10° C. and NMM (1.52 ml, 13.8 mmol) was added followed by H-Pro-OMe*HCl (2.29 g, 13.8 mmol). The mixture was allowed to reach room temperature and left overnight. After removing the solvent and the usual workup the resulting ester 1 was taken without further characterisation. The ester 1 was dissolved in HCl/HOAc (5 ml, 6N) and left at 0° C. until the removal of the Boc-group was complete. The solvent was then removed and the resulting oil was treated with diethylether to give a white solid 2.

Yield: 2.5 g, 80%

Z-Ala-Val-Pro-OMe 3

Z-Ala OH (3.5 g, 15.7 mmol) and 2 (4.18 g, 15.7 mmol) where treated in the same manner as above for 1, to give 3 as a white solid.

Yield: 4.2 g, 64%

Z-Ala-Val-Pro-OH 4

3 (4.2 g, 9.6 mmol) was dissolved in 30 ml of water/acetone (1/5 v/v) and 11.6 ml NaOH (1N) where added. After completion of the reaction the organic solvent was removed by evaporation and the resulting solution was diluted by 15 ml NaHCO₃ solution (saturated). Then the mixture was extracted three times by 10 ml of acetic acid ethyl ester. After that the solution was brought to pH2 by adding HCl (15% in water). The resulting mixture was extracted three times by 30 ml of acetic acid ethyl ester. The organic layer was separated and washed three times with brine, dried (Na₂SO₄) and evaporated.

Yield: 3.5 g, 87%

Z-Ala-Val-Pro-CH₂—Br 5

4 (2.00 g, 4.76 mmol) was dissolved in 15 ml of dry THF and converted into a mixed anhydride (see compound 1) using CAIBE (0.623 ml, 4.76 mmol) and NMM (0.525 ml, 4.76 mmol). The precipitate formed was filtered off and cooled down to −15° C. Then diazomethane (23.8 mmol in 30 ml ether) was dropped into the solution under an argon atmosphere. After leaving the mixture for 1 h at 0° C. 1.27 ml of HBr (33% in AcOH) was added and the solution was stirred for 30 min at room temperature. After that 70 ml of ether was added and the mixture washed with 20 ml of water. The organic layer was separated and dried (Na₂SO₄) and evaporated.

Yield (crude): 1.8 g, 80%

Z-protected acyloxymethylene ketones

The acid (2 eq) was dissolved in DMF and an equimolar amount of KF was added. The suspension was allowed to stir at room temperature for 1 hour. Then the brommethylene (1 eq) component was added and the solution was allowed to stir overnight. After that the solvent was removed under vacuum and the resulting oil was dissolved in chloroform and washed with brine. Then the organic layer was separated dried (Na₂SO₄) and the solvent was removed. The product was purified by column chromatography using silica gel and heptane/chloroform.

Z-Ala-Val-Pro-CH₂O—C(O)—CH₃ 6

Acetic acid (230 μl, 4.02 mmol), KF (0.234 g, 4.02 mmol), 5 (1.00 g, 2.01 mmol)

Yield: 0.351 g, 36%

Z-Ala-Val-Pro-CH₂O—C(O)-Ph 7

Benzoic acid (0.275 g, 2.25 mmol), KF (0.131 mg, 2.25 mmol), 5 (0.56 g. 1.13 mmol)

Yield: 0.34 g, 56%

Deprotection

The Z-protected compound was dissolved in HBr/AcOH and stirred. When the reaction was complete ether was added, the white precipitate formed was filtered off and dried.

H-Ala-Val-Pro-CH₂O—C(O)CH₃*HBr 8

6 (0.351 g, 0.73 mmol)

Yield: 0.252 g, 98%

H-Ala-Val-Pro-CH₂O—C(O)Ph*HBr 9

7 (0.34 g, 0.63 mmol)

Yield: 0.251 g, 99%

### Example 5

### Synthesis of Cycloalkylketones

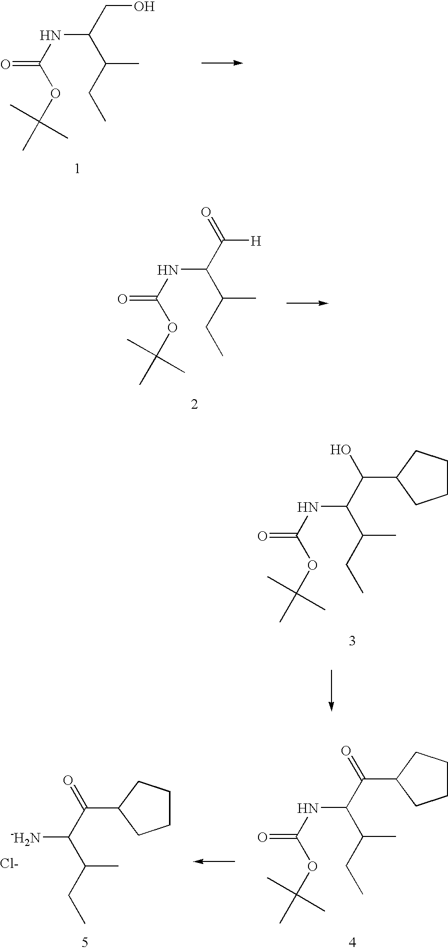 Boc-isoleucinal 2
Oxalylchloride (714 μl, 8.28 mmol) was dissolved in 10 ml of dry dichlormethane and brought to −78° C. Then DMSO (817 μl, 8.28 mmol) was added dropwise. The solution was stirred for 20 min at −78° C. Then 1 (1.00 g, 4.6 mmol) was added and the mixture was stirred for 20 min. After that TEA (2.58 ml, 18.4 mmol) was added and the mixture was allowed to reach room temperature. The mixture was diluted with hexane/ethylacetate (2/1 v/v) and 10 ml of HCl (10% in water) was added. The organic layer was separated and the aqueous phase was extracted with 20 ml of methylenechloride. All organic layers were collected and washed with brine, followed by water, then dried. The product was purified by column chromatography using silica gel and heptane/chloroform.

Yield: 0.52 g, 52%

tert-butyl N-1-[cyclopentyl(hydroxy)methyl]-2-methylbutylcarbamate 3

2 (0.52 g, 2.42 mmol) was dissolved in 10 ml of dry THF and cooled down to 0° C. Then cyclopentylmagnesiumbromide (1.45 ml of a 2 M solution) was added. After completion of the reaction (2 ml) of water was added and solution was neutralized by adding aqueous HCl. Then methylenechloride was added and the organic layer was separated and dried (Na₂SO₄). After evaporation the resulting oil was used without further characterisation.

tert-butyl N-[1-(cyclopentylcarbonyl)-2-methylbutyl]carbamate 4

3 (0.61 g, 2.15 mmol) was treated like 1. Oxalylchloride (333 μl, 3.87 mmol), DMSO (382 μl, 5.37 mmol), TEA (1.2 ml, 8.59 mmol)

Yield: 0.180 g, 30%

1-cyclopentyl-3-methyl-1-oxo-2-pentanaminium chloride 5

4 (0.18 g, 0.63 mmol) was dissolved in 2 ml HCl (7 N in dioxane). After completion of the reaction the solvent was removed and the resulting oil was purified by column chromatography on silical gel using a chloroform/methanol/water gradient. The resulting oil was triturated with ether.

Yield: 0.060 g, 54%

### Example 6

### Synthesis of Side Chain Modified DPIV-Inhibitors

6.1 Synthesis of Boc-glutamyl-thiazolidine (Boc-Glu-Thia)

Reaction of Boc-Glu(OMe)-OH with Thia*HCl according to Method B (see section 6.4 for methods), hydrolysis of Boc-Glu(OMe)-Thia according to Method G

6.1.1 Analytical data for Boc-Glu-Thia

| | Empirical | | | | |
|---|---|---|---|---|---|
| | formula | | | | |
| | Mᵣ | MS [M+H]⁺ | | Elemental | |
| | Synthesis | TLC: | [α]²⁰D | analysis | HPLC |
| | method | R_{f}/system | Concentration | (calc./ | Rₜ |
| Compound | Yield | m.p. | Solvent | found) % | [min]/system |
| Boc-Glu- | C₁₃H₂₂N₂O₅S | 319.5 | −3.1 | C: 49.04/48.89 | 13.93/A² |
| Thia | 318.38 | 0.52/A¹ | c = 1 | H: 6.96/6.82 | |
| | B + G | 0.42/B¹ | methanol | N: 8.80/8.59 | |
| | 62% | 115-118° C. | | | |

¹Thin-layer chromatography
System A: chloroform/methanol 90:10
System B: benzene/acetone/acetic acid 25:10:0.5
System C: n-butanol/EA/acetic acid/H₂O 1:1:1:1
²HPLC separation conditions
Column: Nucleosil C-18, 7μ, 250 mm × 21 mm
Eluant: isocratic, 40% ACN/water/0.1% TFA
Flow rate: 6 ml/min
λ = 220 nm

6.2 Side Chain-Modified Boc-glutamyl Thiazolidines

Boc-Glu-Thia was modified at the γ-carboxylic acid function by introducing radicals of varying size. The radicals were coupled by way of their amino group by forming an amide bond to the γ-carboxylic acid function, with a variety of coupling methods being used depending on the radical. The following amino components were attached to Boc-Glu-Thia using the method stated:

| | Coupling methods | |
|---|---|---|
| Amino component | (see section 3.4) | Yields |
| Polyethylene glycol amine (Mᵣ ≈ | C | 93% |
| 8000) | | |
| H-Gly-Gly-Gly-OH | D + E | 49% |
| H-Gly-Gly-Gly-Gly-Gly-OH | D + E | 86% |

In 2 cases, purification of the reaction products differs from the general description of synthesis.

Boc-Glu(Gly₅)-Thia

The product already precipitates out from the mixture on stirring overnight; it is subsequently filtered off and washed with 0.1N HCl and copious amounts of water and then dried over P₄O₁₀ in vacuo.

Boc-Glu(PEG)-Thia

In contrast to the general procedure, the starting materials for the synthesis are dissolved in a 500-fold excess of DMF. After the reaction is complete, the DMF is completely removed in vacuo and the residue is dissolved in a large amount of methanol. After ether is poured on, to form an upper layer, the product precipitates out together with the unreacted PEG. Fine purification was carried out by preparative HPLC separation on a gel filtration column (Pharmazia, Sephadex G-25, 90 μm, 260 mm-100 mm).

Separating conditions: eluant: water; flow rate: 5 ml/min; λ=220 nm

6.2.2 Synthesis data for side chain-modified Boc-glutamyl thiazolidines

| | Empirical | MS [M+H]⁺ | | Elemental | |
|---|---|---|---|---|---|
| | formula | TLC/R_{f}/ | [α]²⁰D | analysis | HPLC |
| | Mᵣ | system | Concentration | (calc./ | Rₜ |
| Compound | Yield | m.p. | Solvent | found) % | [min]/system |
| Boc- | C₁₉H₃₁N₅O₈S | 490.5 | | C: 46.62 | |
| Glu(Gly₃)- | 489.54 | | | H: 6.38 | |
| Thia | 49% | | | N: 14.31 | |
| Boc- | C₂₃H₃₇N₇O₁₀S | 604.5 | n.dm. | C: 45.76/45.60 | 11.93/A² |
| Glu(Gly₅)- | 603.64 | 0.09/C | | H: 6.18/6.11 | |
| Thia | 86% | decomp. | | N: 16.24/16.56 | |
| | | from 202° C. | | | |
| Boc- | 93% | ≈ 8000 | n.dm. | n.dm. | n.dm. |
| Glu(PEG)- | | (mass | | | |
| Thia | | emphasis) | | | |
| | | 52-53° C. | | | |

²HPLC separation conditions
Column: Nucleosil C-18, 7μ, 250 mm × 21 mm
Eluant: isocratic, 40% ACN/water/0.1% TFA
Flow rate: 6 ml/min
λ = 220 nm

6.3 Side Chain-Modified Glutamyl Thiazolidines

The N-terminal Boc protecting groups were cleaved off the compounds described in Table 6.2.2 using method F. The substances modified with Gly derivatives were purified by preparative HPLC separation and are present as trifluoroacetates. The H-Glu(PEG)-Thia was purified on a gel filtration column in the same manner as the Boc-protected precursor.

6.3.1 Synthesis data for side chain-modified glutamyl thiazolidines

| | Empirical | MS [M+H]⁺ | | Elemental | |
|---|---|---|---|---|---|
| | formula | TLC/R_{f}/ | [α]²⁰D | analysis | HPLC |
| | Mᵣ | system | Concentration | (calc./ | Rₜ [min]/ |
| Compound | Yield | m.p. | Solvent | found) % | system |
| H- | C₁₆H₂₄N₅O₈SF₃ | 503.45 | +4.1 | C: 38.17/37.56 | 7.84/C³ |
| Glu(Gly₃)- | 503.45 | 0.32/C | c = 1 | H: 4.80/4.78 | |
| Thia *TFA | 94% | 91-94° C. | methanol | N: 13.91/13.43 | |
| H- | C₂₀H₃₀N₇O₁₀SF₃ | 617.55 | n.dm. | C: 38.90/38.82 | 8.22/C³ |
| Glu(Gly₅)- | 617.55 | 0.25/C | | H: 4.90/4.79 | |
| Thia *TFA | 98% | 105-107° C. | | N: 15.88/15.39 | |
| H- | 92% | ≈ 8000 | n.dm. | n.dm. | n.dm. |
| Glu(PEG)- | | (mass | | | |
| Thia *HCl | | emphasis) | | | |

³HPLC separation conditions
Column: Nucleosil C-18, 7μ, 250 mm × 21 mm
Eluant: ACN/water/0.1% TFA
Gradient: 20% ACN → 90% ACN over 30 min
Flow rate: 6 ml/min
λ = 220 nm
n.dm. — not determined or not determinable

6.4 General Synthesis Procedures
Method A: Peptide Bond Attachment by the Mixed Anhydride Method Using CFIBE as Activation Reagent

10 mmol of N-terminally protected amino acid or peptide are dissolved in 20 ml of absolute THF. The solution is cooled to −15° C.±2° C. With stirring in each case, 10 mmol of N-MM and 10 mmol of chloroformic acid isobutyl ester are added in succession, the stated temperature range being strictly adhered to. After approximately 6 min, 10 mmol of the amino component is added. When the amino component is a salt, a further 10 mmol of N-MM is then added to the reaction mixture. The reaction mixture is then stirred for 2 h in the cold state and overnight at room temperature.

The reaction mixture is concentrated using a rotary evaporator, taken up in EA, washed with 5% KH₂SO₄ solution, saturated NaHCO₃ solution and saturated NaCl solution and dried over NaSO₄. After removal of the solvent in vacuo, the compound is recrystallized from EA/pentane.

Method B: Peptide Bond Attachment by the Mixed Anhydride Method Using Pivalic Acid Chloride as Activation Reagent

10 mmol of N-terminally protected amino acid or peptide are dissolved in 20 ml of absolute THF. The solution is cooled to 0° C. With stirring in each case, 10 mmol of N-MM and 10 mmol of pivalic acid chloride are added in succession, the stated temperature range being strictly adhered to. After approximately 6 min, the mixture is cooled to −15° C. and, once the lower temperature has been reached, 10 mmol of the amino component is added. When the amino component is a salt, a further 10 mmol of N-MM is then added to the reaction mixture. The reaction mixture is then stirred for 2 h in the cold state and overnight at room temperature.

Further working up is carried out as in Method A.

Method C: Peptide Bond Attachment Using TBTU as Activation Reagent

10 mmol of the N-terminally protected amino acid or peptide and 10 mmol of the C-terminally protected amino component are dissolved in 20 ml of absolute DMF. The solution is cooled to 0° C. With stirring in each case, 10 mmol of DIPEA and 10 mmol of TBTU are added in succession. The reaction mixture is stirred for one hour at 0° C. and then overnight at room temperature. The DMF is completely removed in vacuo and the product is worked up as described in Method A.

Method D: Synthesis of an Active Ester (N-hydroxysuccinimide Ester)

10 mmol of N-terminally protected amino acid or peptide and 10 mmol of N-hydroxy-succinimide are dissolved in 20 ml of absolute THF. The solution is cooled to 0° C. and 10 mmol of dicyclohexylcarbodiimide are added, with stirring. The reaction mixture is stirred for a further 2 h at 0° C. and then overnight at room temperature. The resulting N,N′-dicyclohexylurea is filtered off and the solvent is removed in vacuo and the remaining product is recrystallized from EA/pentane.

Method E: Amide Bond Attachment Using N-hydroxysuccinimide Esters

10 mmol of the C-terminally unprotected amino component is introduced into an NaHCO₃ solution (20 mmol in 20 ml of water). At room temperature and with stirring, 10 mmol of the N-terminally protected N-hydroxysuccinimide ester dissolved in 10 ml of dioxane are slowly added dropwise. Stirring of the reaction mixture is continued overnight and the solvent is then removed in vacuo.

Further working up is carried out as in Method A.

Method F: Cleavage of the Boc Protecting Group

3 ml of 1.1N HCl/glacial acetic acid (Method F1) or 3 ml of 1.1N HCl/dioxane (Method F2) or 3 ml of 50% TFA in DCM (Method F3) are added to 1 mmol of Boc-protected amino acid pyrrolidide, thiazolidide or peptide. The cleavage at RT is monitored by means of TLC. After the reaction is complete (approximately 2 h), the compound is precipitated in the form of the hydrochloride using absolute diethyl ether and is isolated with suction and dried over P₄O₁₀ in vacuo. Using methanol/ether, the product is recrystallized or reprecipitated.

Method G: Hydrolysis

1 mmol of peptide methyl ester is dissolved in 10 ml of acetone and 11 ml of 0.1M NaOH solution and stirred at room temperature. The course of the hydrolysis is monitored by means of TLC. After the reaction is complete, the acetone is removed in vacuo. The remaining aqueous solution is acidified, using concentrated KH₂SO₄ solution, until a pH of 2-3 is reached. The product is then extracted several times using EA; the combined ethyl acetate fractions are washed with saturated NaCl solution and dried over NaSO₄, and the solvent is removed in vacuo. Crystallization from EA/pentane is carried out.

### Example 7

### Kᵢ-Determination

For Kᵢ determination, dipeptidyl peptidase IV from porcine kidney with a specific activity against glycylprolyl-4-nitroaniline of 37.5 U/mg and an enzyme concentration of 1.41 mg/ml in the stock solution was used.

Assay Mixture:

100 μl test compound in a concentration range of 1*10⁻⁵ M-1*10⁻⁸ M respectively were admixed with 50 μl glycylprolyl-4-nitroaniline in different concentrations (0.4 mM, 0.2 mM, 0.1 mM, 0.05 mM) and 100 μl HEPES (40 mM, pH7.6; ion strength=0.125). The assay mixture was pre-incubated at 30° C. for 30 min. After pre-incubation, 20 μl DPIV (1:600 diluted) was added and measurement of yellow color development due to 4-nitroaniline release was performed at 30° C. and λ=405 nm for 10 min. using a plate reader (HTS7000 plus, Applied Biosystems, Weiterstadt, Germany).

The Kᵢ-values were calculated using Graphit version 4.0.13, 4.0.13 and 4.0.15 (Erithacus Software, Ltd, UK).

7.1 Results - Ki values of DPIV inhibition

| Compound | Ki [M] |
|---|---|
| H-Asn-pyrrolidine | 1.20 * 10⁻⁵ |
| H-Asn-thiazolidine | 3.5 * 10⁻⁶ |
| H-Asp-pyrrolidine | 1.4 * 10⁻⁵ |
| H-Asp-thiazolidine | 2.9 * 10⁻⁶ |
| H-Asp-(NHOH)-pyrrolidine | 1.3 * 10⁻⁵ |
| H-Asp-(NHOH)-thiazolidine | 8.8 * 10⁻⁶ |
| H-Glu-pyrrolidine | 2.2 * 10⁻⁶ |
| H-Glu-thiazolidine | 6.1 * 10⁻⁷ |
| H-Glu(NHOH)-pyrrolidine | 2.8 * 10⁻⁶ |
| H-Glu(NHOH)-thiazolidine | 1.7 * 10⁻⁶ |
| H-His-pyrrolidine | 35 * 10⁻⁶ |
| H-His-thiazolidine | 1.8 * 10⁻⁶ |
| H-Pro-pyrrolidine | 4.1 * 10⁻⁶ |
| H-Pro-pyrrolidine | 1.2 * 10⁻⁶ |
| H-Ile-azididine | 3.1 * 10⁻⁶ |
| H-Ile-pyrrolidine | 2.1 * 10⁻⁷ |
| H-L-threo-Ile-thiazolidine | 8.0 * 10⁻⁸ |
| H-L-allo-Ile-thiazolidine | 1.9 * 10⁻⁷ |
| D-threo-isoleucyl-thiazolidine-fumarate | no inhibition |
| D-allo-isoleucyl-thiazolidine-fumarate | no inhibition |
| H-L-threo-Ile-thiazolidine-succinate | 5.1 * 10⁻⁸ |
| H-L-threo-Ile-thiazolidine-tartrate | 8.3 * 10⁻⁸ |
| H-L-threo-Ile-thiazolidine-fumarate | 8.3 * 10⁻⁸ |
| H-L-threo-Ile-thiazolidine-hydrochloride | 7.2 * 10⁻⁸ |
| H-L-threo-Ile-thiazolidine-phosphate | 1.3 * 10⁻⁷ |
| H-Val-pyrrolidine | 4.8 * 10⁻⁷ |
| H-Val-thiazolidine | 2.7 * 10⁻⁷ |
| Diprotin A | 3.45 * 10⁻⁶ |
| Diprotin B | 2.24 * 10⁻⁵ |
| Nva-Pro-Ile | 6.17 * 10⁻⁶ |
| Cha-Pro-Ile | 5.99 * 10⁻⁶ |
| Nle-Pro-Ile | 9.60 * 10⁻⁶ |
| Phe-Pro-Ile | 1.47 * 10⁻⁵ |
| Val-Pro-Val | 4.45 * 10⁻⁶ |
| Ile-Pro-Val | 5.25 * 10⁻⁶ |
| Abu-Pro-Ile | 8.75 * 10⁻⁶ |
| Ile-Pro-allo-Ile | 5.22 * 10⁻⁶ |
| Val-Pro-allo-Ile | 9.54 * 10⁻⁶ |
| Tyr-Pro-allo-Ile | 1.82 * 10⁻⁵ |
| AOA-Pro-Ile | 1.26 * 10⁻⁵ |
| t-butyl-Gly-Pro-Ile | 3.10 * 10⁻⁶ |
| Ser(Bzl)-Pro-Ile | 2.16 * 10⁻⁵ |
| Aze-Pro-Ile | 2.05 * 10⁻⁵ |
| t-butyl-Gly-Pro-Val | 3.08 * 10⁻⁶ |
| Gln-Pyrr | 2.26 * 10⁻⁶ |
| Gln-Thia | 1.21 * 10⁻⁶ |
| Val-Pro-t-butyl-Gly | 1.96 * 10⁻⁵ |
| t-butyl-Gly-Pro-Gly | 1.51 * 10⁻⁵ |
| Ile-Pro-t-butyl-Gly | 1.89 * 10⁻⁵ |
| t-butyl-Gly-Pro-IleNH₂ | 5.60 * 10⁻⁶ |
| t-butyl-Gly-Pro-D-Val | 2.65 * 10⁻⁵ |
| t-butyl-Gly-Pro-t-butyl-Gly | 1.41 * 10⁻⁵ |
| Ile-cyclopentyl ketone | 6.29 * 10⁻⁶ |
| t-butyl-Gly-cyclohexyl ketone | 2.73 * 10⁻⁴ |
| Ile-cyclohexyl ketone | 5.68 * 10⁻⁵ |
| Val-cyclopentyl ketone | 1.31 * 10⁻⁵ |
| Val-Pro-methyl ketone | 4.76 * 10⁻⁸ |
| Val-Pro-acyloxy methyl ketone | 1.05 * 10⁻⁹ |
| Val-Pro-benzoyl methyl ketone | 5.36 * 10⁻¹⁰ |
| Val-Pro-benzothiazol methyl ketone | 3.73 * 10⁻⁸ |
| H-Glu-Thia | 6.2 * 10⁻⁷ |
| H-Gly(NHOH)-Thia | 1.7 * 10⁻⁶ |
| H-Glu(Gly₃)-Thia | 1.92 * 10⁻⁸ |
| H-Glu(Gly₅)-Thia | 9.93 * 10⁻⁸ |
| H-Glu(PEG)-Thia | 3.11 * 10⁻⁶ |

t-butyl-Gly is defined as: 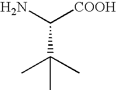
Ser(Bzl) and Ser(P) are defined as benzyl-serine and phosphoryl-serine, respectively. Tyr(P) is defined as phosphoryl-tyrosine.

### Example 8

### Determination of IC₅₀-Values

100 μl inhibitor stock solution were mixed with 100 μl buffer (HEPES pH7.6) and 50 μl substrate (Gly-Pro-pNA, final concentration 0.4 mM) and preincubated at 30° C. Reaction was started by addition of 20 μl purified porcine DPIV. Formation of the product pNA was measured at 405 nm over 10 min using the HTS 7000Plus plate reader (Perkin Elmer) and slopes were calculated. The final inhibitor concentrations ranged between 1 mM and 30 nM. For calculation of IC50 GraFit 4.0.13 (Erithacus Software) was used.

8.1 Results - Determination of IC₅₀ values

| Compound | IC50 [M] |
|---|---|
| Isoleucyl thiazolidine fumarate | 1.28 * 10⁻⁷ |
| Diprotin A | 4.69 * 10⁻⁶ |
| Diprotin B | 5.54 * 10⁻⁵ |
| Phg-Pro-Ile | 1.54 * 10⁻⁴ |
| Nva-Pro-Ile | 2.49 * 10⁻⁵ |
| Cha-Pro-Ile | 2.03 * 10⁻⁵ |
| Nle-Pro-Ile | 2.19 * 10⁻⁵ |
| Ser(P)-Pro-Ile | 0.012 |
| Tyr(P)-Pro-Ile | 0.002 |
| Phe-Pro-Ile | 6.20 * 10⁻⁵ |
| Trp-Pro-Ile | 3.17 * 10⁻⁴ |
| Ser-Pro-Ile | 2.81 * 10⁻⁴ |
| Thr-Pro-Ile | 1.00 * 10⁻⁴ |
| Val-Pro-Val | 1.64 * 10⁻⁵ |
| Ile-Pro-Val | 1.52 * 10⁻⁵ |
| Abu-Pro-Ile | 3.43 * 10⁻⁵ |
| Pip-Pro-Ile | 0.100 |
| Ile-Pro-allo-Ile | 1.54 * 10⁻⁵ |
| Val-Pro-allo-Ile | 1.80 * 10⁻⁵ |
| Tyr-Pro-allo-Ile | 6.41 * 10⁻⁵ |
| AOA-Pro-Ile | 4.21 * 10⁻⁵ |
| t-butyl-Gly-Pro-Ile | 9.34 * 10⁻⁶ |
| Ser(Bzl)-Pro-Ile | 6.78 * 10⁻⁵ |
| Tic-Pro-Ile | 0.001 |
| Orn-Pro-Ile | 2.16 * 10⁻⁴ |
| Gln-Thia | 5.27 * 10⁻⁶ |
| Aze-Pro-Ile | 7.28 * 10⁻⁵ |
| Ile-Hyp-Ile | 0.006 |
| t-butyl-Gly-Pro-Val | 1.38 * 10⁻⁵ |
| Gln-Pyrr | 1.50 * 10⁻⁵ |
| Val-Pro-t-butyl-Gly | 6.75 * 10⁻⁵ |
| t-butyl-Gly-Pro-Gly | 5.63 * 10⁻⁵ |
| Ile-Pro-t-butyl-Gly | 8.23 * 10⁻⁵ |
| t-butyl-Gly-Pro-IleNH₂ | 2.29 * 10⁻⁵ |
| t-butyl-Gly-Pro-D-Val | 1.12 * 10⁻⁴ |
| t-butyl-Gly-Pro-t-butyl-Gly | 2.45 * 10⁻⁵ |
| Aib-Pro-Ile | no inhibition |
| Ile-cyclopentyl ketone | 3.82 * 10⁻⁵ |
| t-butyl-Gly-cyclohexyl ketone | 2.73 * 10⁻⁴ |
| Ile-cyclohexyl ketone | 2.93 * 10⁻⁴ |
| Val-cyclopentyl ketone | 4.90 * 10⁻⁵ |
| Val-cyclohexyl ketone | 0.001 |
| Val-Pro-methyl ketone | 5.79 * 10⁻⁷ |
| Val-Pro-acyloxy methyl ketone | 1.02 * 10⁻⁸ |
| Val-Pro-benzoyl methyl ketone | 1.79 * 10⁻⁸ |
| Val-Pro-benzothiazol methyl ketone | 1.38 * 10⁻⁷ |

t-butyl-Gly is defined as: 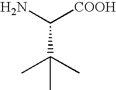
Ser(Bzl) and Ser(P) are defined as benzyl-serine and phosphoryl-serine, respectively. Tyr(P) is defined as phosphoryl-tyrosine.

### Example 9

### Inhibition of DPIV-Like Enzymes—Dipeptidyl Peptidase II

DP II (3.4.14.2) releases N-terminal dipeptides from oligopeptides if the N-terminus is not protonated (McDonald, J. K., Ellis, S. & Reilly, T. J., 1966, J. Biol. Chem., 241, 1494-1501). Pro and Ala in P₁-position are preferred residues. The enzyme activity is described as DPIV-like activity, but DP II has an acidic pH-optimum. The enzyme used was purified from porcine kidney.

Assay:

100 μl glutaminyl pyrrolidine or glutaminyl thiazolidine in an concentration range of 1*10⁻⁴M-5*10⁻⁸M were admixed with 100 μl μl buffer solution (40 mM HEPES, pH7.6, 0.015% Brij, 1 mM DTT), 50 μl lysylalanylaminomethylcoumarine solution (5 mM) and 20 μl porcine DP II (250 fold diluted in buffer solution). Fluorescence measurement was performed at 30° C. and λₑₓᵢₐₜₐₜᵢₒₙ=380 nm, λₑₘᵢₛₛᵢₒₙ=465 nm for 25 min using a plate reader (HTS7000plus, Applied Biosystems, Weiterstadt, Germany). The Kᵢ-values were calculated using Graphit 4.0.15 (Erithacus Software, Ltd., UK) and were determined as Kᵢ=8.52*10⁻⁵ M±6.33*10⁻⁶ M for glutaminyl pyrrolidine and Kᵢ=1.07*10⁻⁵ M±3.81*10⁻⁷ M for glutaminyl thiazolidine.

### Example 10

### Cross Reacting Enzymes

Glutaminyl pyrrolidine and glutaminyl thiazolidine were tested for their cross reacting potency against dipeptidyl peptidase I, prolyl oligopeptidase and prolidase.

Dipeptidyl Peptidase I (DP I, Cathepsin C):

DP I or cathepsin C is a lysosomal cysteine protease which cleaves off dipeptides from the N-terminus of their substrates (Gutman, H. R. & Fruton, J. S., 1948, J. Biol: Chem., 174, 851-858). It is classified as a cysteine protease. The enzyme used was purchased from Qiagen (Qiagen GmbH, Hilden, Germany). In order to get a fully active enzyme, the enzyme was diluted 1000 fold in MES buffer pH5.6 (40 mM MES, 4 mM DTT, 4 mM KCl, 2 mM EDTA, 0.015% Brij) and pre-incubated for 30 min at 30° C.

Assay:

50 μl glutaminyl pyrrolidine or glutaminyl thiazolidine in a concentration range of 1*10⁻⁵ M-1*10⁻⁷M were admixed with 110 μl buffer-enzyme-mixture. The assay mixture was pre-incubated at 30° C. for 15 min. After pre-incubation, 100 μl histidylseryl-□-nitroaniline (2*10⁻⁵M) was added and measurement of yellow color development due to β-nitroaniline release was performed at 30° C. and λ_{excitation}=380 nm, λₑₘᵢₛₛᵢₒₙ=465 nm for 10 min., using a plate reader (HTS7000 plus, Applied Biosystems, Weiterstadt, Germany).

The IC₅₀-values were calculated using Graphit 4.0.15 (Erithacus Software, Ltd., UK). No inhibition of the DP I enzyme activity by glutaminyl pyrrolidine or glutaminyl thiazolidine was found.

Prolyl Oligopeptidase (POP)

Prolyl oligopeptidase (EC 3.4.21.26) is a serine type endoprotease which cleaves off peptides at the N-terminal part of the Xaa-Pro bond (Walter, R., Shlank, H., Glass, J. D., Schwartz, I. L. & Kerenyi, T. D., 1971, Science, 173, 827-829). Substrates are peptides with a molecular weight up to 3000 Da. The enzyme used was a recombinant human prolyl oligopeptidase. Recombinant expression was performed in E. coli under standard conditions as described elsewhere in the state of the art.

Assay:

100 μl glutaminyl pyrrolidine or glutaminyl thiazolidine in an concentration range of 1*10⁻⁴M-5*10⁻⁸ M were admixed with 100 μl μl buffer solution (40 mM HEPES, pH7.6, 0.015% Brij, 1 mM DTT) and 20 μl POP solution. The assay mixture was pre-incubated at 30° C. for 15 min. After pre-incubation, 50 μl glycylprolylprolyl-4-nitroaniline solution (0.29 mM) was added and measurement of yellow color development due to 4-nitroaniline release was performed at 30° C. and λ=405 nm for 10 min using a plate reader (sunrise, Tecan, Crailsheim, Germany). The IC₅₀-values were calculated using Graphit 4.0.15 (Erithacus Software, Ltd., UK). No inhibition of POP activity by glutaminyl pyrrolidine or glutaminyl thiazolidine was found.

Prolidase (X-Pro Dipeptidase)

Prolidase (EC 3.4.13.9) was first described by Bergmann & Fruton (Bergmann, M. & Fruton, J S, 1937, J. Biol. Chem. 189-202). Prolidase releases the N-terminal amino acid from Xaa-Pro dipeptides and has a pH optimum between 6 and 9.

Prolidase from porcine kidney (ICN Biomedicals, Eschwege, Germany) was solved (1 mg/ml) in assay buffer (20 mM NH₄(CH₃COO)₂, 3 mM MnCl₂, pH 7.6). In order to get a fully active enzyme the solution was incubated for 60 min at room temperature.

Assay:

450 μl glutaminyl pyrrolidine or glutaminyl thiazolidine in an concentration range of 5*10⁻³ M-5*10⁻⁷ M were admixed with 500 μl buffer solution (20 mM NH₄(CH₃COO)₂, pH 7.6) and 250 μl Ile-Pro-OH (0.5 mM in the assay, mixture). The assay mixture was pre-incubated at 30° C. for 5 min. After pre-incubation, 75 μl Prolidase (1:10 diluted in assay buffer) were added and measurement was performed at 30° C. and λ=220 nm for 20 min using a UV/Vis photometer, UV1 (Thermo Spectronic, Cambridge, UK).

The IC 50-values were calculated using Graphit 4.0.15 (Erithacus Software, Ltd., UK). They were determined as IC₅₀>3 mM for glutaminyl thiazolidine and as IC₅₀=3.4*10⁻⁴M±5.63*10⁻⁵ for glutaminyl pyrrolidine.

### Example 11

### Determination of DPIV Inhibiting Activity After Intravasal and Oral Administration to Wistar Rats

Animals

Male Wistar rats (Shoe: Wist(Sho)) with a body weight ranging between 250 and 350 g were purchased from Tierzucht Schönwalde (Schönwalde, Germany).

Housing Conditions

Animals were single-caged under conventional conditions with controlled temperature (22±2° C.) on a 12/12 hours light/dark cycle (light on at 06:00 AM). Standard pelleted chow (ssniff® Soest, Germany) and tap water acidified with HCl were allowed ad libitum.

Catheter Insertion into Carotid Artery

After ≧one week of adaptation at the housing conditions, catheters were implanted into the carotid artery of Wistar rats under general anaesthesia (i.p. injection of 0.25 ml/kg b.w. Rompun® [2%], BayerVital, Germany and 0.5 ml/kg b.w. Ketamin 10, Atarost GmbH & Co., Twistringen, Germany). The animals were allowed to recover for one week. The catheters were flushed with heparin-saline (100 IU/ml) three times per week. In case of catheter dysfunction, a second catheter was inserted into the contra-lateral carotid artery of the respective rat. After one week of recovery from surgery, this animal was reintegrated into the study. In case of dysfunction of the second catheter, the animal was withdrawn from the study. A new animal was recruited and the experiments were continued in the planned sequence, beginning at least 7 days after catheter implantation.

Experimental Design

Rats with intact catheter function were administered placebo (1 ml saline, 0.154 mol/l) or test compound via the oral and the intra-vasal (intra-arterial) route. After overnight fasting, 100 μl samples of heparinised arterial blood were collected at −30, −5, and 0 min. The test substance was dissolved freshly in 1.0 ml saline (0.154 mol/l) and was administered at 0 min either orally via a feeding tube (75 mm; Fine Science Tools, Heidelberg, Germany) or via the intra-vasal route. In the case of oral administration, an additional volume of 1 ml saline was injected into the arterial catheter. In the case of infra-arterial administration, the catheter was immediately flushed with 30 μl saline and an additional 1 ml of saline was given orally via the feeding tube.

After application of placebo or the test substances, arterial blood samples were taken at 2.5, 5, 7.5, 10, 15, 20, 40, 60 and 120 min from the carotid catheter of the conscious unrestrained rats. All blood samples were collected into ice cooled Eppendorf tubes (Eppendorf-Netheler-Hinz, Hamburg, Germany) filled with 10 μl 1M sodium citrate buffer (pH 3.0) for plasma DPIV activity measurement. Eppendorf tubes were centrifuged immediately (12000 rpm for 2 min, Hettich Zentrifuge EBA 12, Tuttlingen; Germany): The plasma fractions were stored on ice until analysis or were frozen at −20° C. until analysis. All plasma samples were labelled with the following data:

Code number

Animal Number

Date of sampling

Time of sampling

Analytical Methods

The assay mixture for determination of plasma DPIV activity consisted of 80 μl reagent and 20 μl plasma sample. Kinetic measurement of the formation of the yellow product 4-nitroaniline from the substrate glycylprolyl-4-nitroaniline was performed at 390 nm for 1 min at 30° C. after 2 min pre-incubation at the same temperature. The DPIV activity was expressed in mU/ml.

Statistical Methods

Statistical evaluations and graphics were performed with PRISM® 3.02 (GraphPad Software, Inc.). All parameters were analysed in a descriptive manner including mean and SD.

11.1 Results - in vivo DPIV-inhibition at tₘₐₓ

| | Dose | | |
|---|---|---|---|
| STRUCTURE | (mg/kg) | i.v. (%) | p.o. (%) |
| Gln-Pyrr | 100 | 80 | 67 |
| Gln-Thia | 100 | 88 | 71 |
| Diprotin A | 100 | 73 | no inhibition |
| Diprotin B | 100 | 50 | no inhibition |
| Tyr(P)-Pro-Ile | 100 | 37 | no inhibition |
| t-butyl-Gly-Pro-Ile | 100 | 71 | 28 |
| t-butyl-Gly-Pro-Val | 100 | 72 | 25 |
| Ala-Val-Pro-acyloxy methyl | 100 | 89 | 86 |
| ketone | | | |
| Ala-Val-Pro-benzoyl- | 100 | 97 | 76 |
| methyl ketone | | | |
| Ile-cyclopentyl ketone | 100 | 34 | 15 |

### Example 12

### Action of Side Chain-Modified Glutamyl Thiazolidines as Non-Readily-Transportable DPIV-Inhibitors

Side chain-modified glutamyl thiazolidines having a structure H-Glu(X)-Thia were synthesised, with polyethylene glycol or glycine oligomers of various chain lengths being used as X (see Method A of example for description of synthesis). The binding characteristics of those derivatives and their transportability by the peptide transporter PepT1 were investigated.

Surprisingly, it was found that the side chain modifications alter the binding characteristics of the compounds to DPIV only to a slight extent. In contrast, the ability of the inhibitors to be transported by the peptide transporter is dramatically diminished by the side chain modification.

Side chain modified inhibitors of DPIV or DPIV-like enzymes are therefore well suited to achieving site directed inhibition of DPIV in the body.

12.1 Results: Transportability of selected DRIV-inhibitors.

| Compound | EC50 (mM)¹ | Iₘₐₓ (nA)² |
|---|---|---|
| amino acid thiazolidines | | |
| H-Ile-Thia | 0.98 | 25 ± 8 |
| H-Glu-Thia | 1.1 | 35 ± 13 |
| side chain-modified | | |
| glutamylthiazolidines | | |
| H-Gly(NHOH)-Thia | 3.18 | 42 ± 11 |
| H-Glu(Gly₃)-Thia | 8.54 | n.d.³ |
| H-Glu(Gly₅)-Thia | >10 | n.d.³ |
| H-Glu(PEG)-Thia | >10 | n.d.³ |

¹Effective concentrations of the compounds inhibiting the binding of ³ H-D-Phe-Ala (80 mM) to PepT1-expressing P. pastoris cells by 50% (EC₅₀ values)
²Transport characteristics at PepT1-expressing oocytes of X. leavis - by means of two-electrode voltage clamp method, I = inward currents generated by the transport

### Example 13

### Inhibition of the DPIV-catalyzed hydrolysis of the incretins GIP₁₋₄₂ and GLP-1₇₋₃₆ in vitro

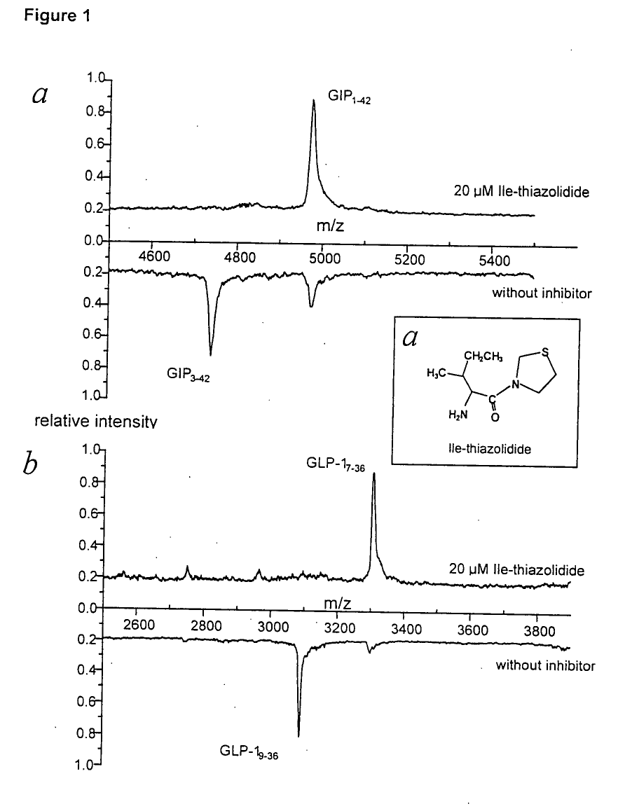

It is possible to suppress the in vitro hydrolysis of incretins caused by DPIV and DPIV-like enzymatic activity using purified enzyme or pooled human serum (FIG. 1).

According to the present invention complete suppression of the enzyme-catalyzed hydrolysis of both peptide hormones is achieved in vitro by incubating 30 mM GIP₁₋₄₂ or 30 mM GLP-1₇₋₃₆ and 20 mM isoleucyl thiazolidine (1a), a reversible DPIV-inhibitor, in 20% of pooled serum at pH 7.6 and 30° C. over 24 hours (1b and 1c, both upper spectra: Synthetic GIP₁₋₄₂ (5 mM) and synthetic GLP-1₇₋₃₆ (15 μM) were incubated with human serum (20%) in 0.1 mM TRICINE Puffer at pH 7.6 and 30° C. for 24 hours. Samples of the incubation assays (in the case of GIP₁₋₄₂ 2.5 pmol and in the case of GLP-1₇₋₃₆ 7.5 pmol) have been withdrawn after different time intervals. Samples were cocrystallized using 2′,6′-dihydroxy-acetophenon as matrix and analyzed by MALDI-TOF-mass spectrometry. Spectra (FIG. 1) display accumulations of 250 single laser shots per sample.

(1b) The signal of m/z 4980.1±5.3 corresponds to the DPIV-substrate GIP₁₋₄₂ (M 4975.6) and the signal of the mass m/z 4745.2±5.5 corresponds to the DPIV-released product GIP3-42 (M 4740.4).

(1c) The signal of m/z 3325.0±1.2 corresponds to the DPIV-substrate GLP-1₇₋₃₆ (M 3297.7) and the signal of mass m/z 3116.7±1.3 to the DPIV-released product GLP-1₉₋₃₆ (M 3089.6).

In the control assays containing no inhibitor the incretins were almost completely degraded (FIGS. 1b and 1c, both bottom spectra).

### Example 14

### Inhibition of the Degradation of GLP1₇₋₃₆ by the DPIV-Inhibitor Isoleucyl Thiazolidine in vivo

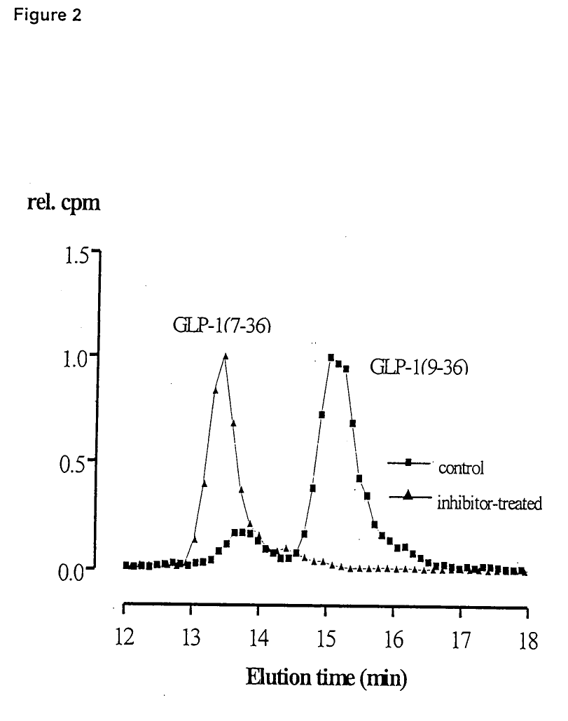

Analysis of the metabolism of native incretins (in this case GLP-1₇₋₃₆) in the circulation of the rat in the presence or absence of the DPIV-inhibitor isoleucyl thiazolidine (i.v. injection of 1.5 M inhibitor in 0.9% saline solution) and of a control. No degradation of the insulinotropic peptide hormone GLP-1₇₋₃₆ occurs at a concentration of 0.1 mg/kg of the inhibitor isoleucyl thiazolidine in treated animals (n=5) during the time course of the experiment (FIG. 2).

To analyze the metabolites of the incretins in the presence and absence of the DPIV-inhibitor, test and control animals received a further i.v. injection of 50-100 pM ¹²⁵I-GLP-1₇₋₃₆ (specific activity about 1 μCi/pM) 20 min after an initial i.v.-inhibitor and/or saline administration. Blood samples were collected after 2-5 min incubation time and the plasma was extracted using 20% acetonitrile. Subsequently, the peptide extract was separated on RP-HPLC. Multiple fractions of eluent were collected between 12-18 min and counted on a γ-counter. Data are expressed as counts per minute (cpm) relative to the maximum.

### Example 15

### Modulation of Insulin Responses and Reduction of the Blood Glucose Level After i.v. Administration of the DPIV-Inhibitor Isoleucyl Thiazolidine in vivo

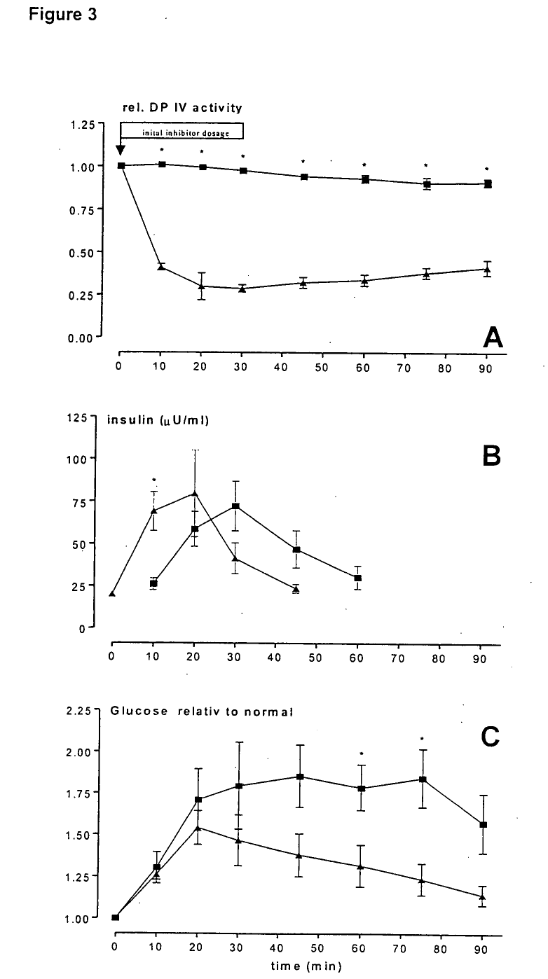

The figure shows circulating glucose and insulin responses to intraduodenal (i.d.) administration of glucose to rats in the presence or absence of isoleucyl thiazolidine (0.1 mg per kg). There is a more rapid reduction in the circulating glucose concentration in animals, which received DPIV-effectors when compared to untreated controls. The observed effect is dose dependent and reversible after termination of an infusion of 0.05 mg/min of the DPIV-inhibitor isoleucyl thiazolidine per kg rat. In contrast to the i.d. glucose-stimulated animals, there was no comparable effect observable after the i.v. administration of the same amount of glucose in inhibitor-treated control animals. In FIG. 3 these relationships are demonstrated displaying the inhibitor-dependent changes of selected plasma parameter: A—DPIV-activity, B—plasma-insulin level, C—blood glucose level.

### Example 16

### Impact of Chronic Treatment of Fatty Zucker Rats on the Fasting Blood Glucose During 12 Weeks of Oral Drug Application

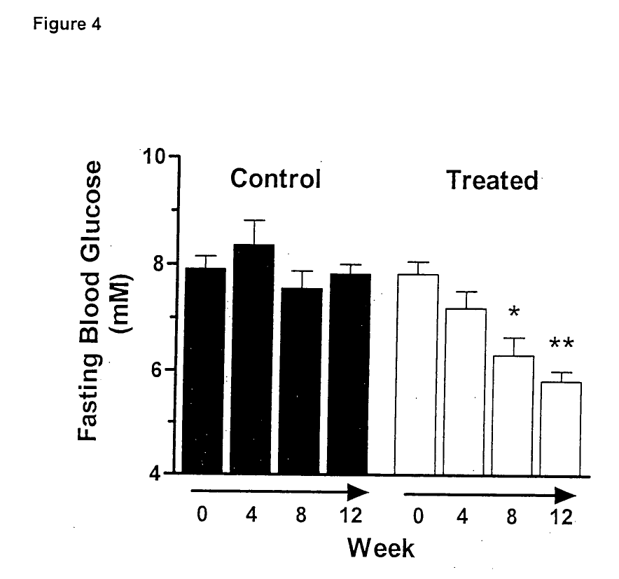

Chronic application of the DPIV-inhibitor isoleucyl thiazolidine fumarate results in dramatic reduction and almost normalization of the fasting blood glucose in the chosen diabetic rat model (FIG. 4).

Animals.

Six pairs of male fatty (fa/fa) VDF Zucker rat littermates were randomly assigned to either a control or treatment (isoleucyl thiazolidine fumarate) group at 440 g body weight (11±0.5 weeks of age). Animals were housed singly, on a 12 hour light/dark cycle (lights on at 6 am) and allowed access to standard rat food, and water ad libitum.

Protocol for Daily Monitoring and Drug Administration.

The treatment group received 10 mg/kg isoleucyl thiazolidine fumarate by oral gavage twice daily (8:00 a.m. and 5:00 p.m.) for 100 days, while the control animals received concurrent doses of vehicle consisting of a 1% cellulose solution. Every two days, body weight, morning and evening blood glucose, and food and water intake were assessed. Blood samples for glucose determination were acquired from tail bleeds, and measured using a SureStep glucose analyzer (Lifescan Canada Ltd., Burnaby).

Protocol for Monthly Assessment of Glucose Tolerance.

Every four weeks from the start of the experiment, an oral glucose tolerance test (OGTT) was performed: animals were fasted for 18 hours following the 1700 h dosing and administered 1 g/kg glucose orally. This time period is equivalent to ˜12 circulating half-lives of isoleucyl thiazolidine fumarate.

### Example 17

### Impact of Chronic Oral Treatment of Fatty Zucker Rats on Systolic Blood Pressure with the DPIV-Inhibitor Isoleucyl Thiazolidine

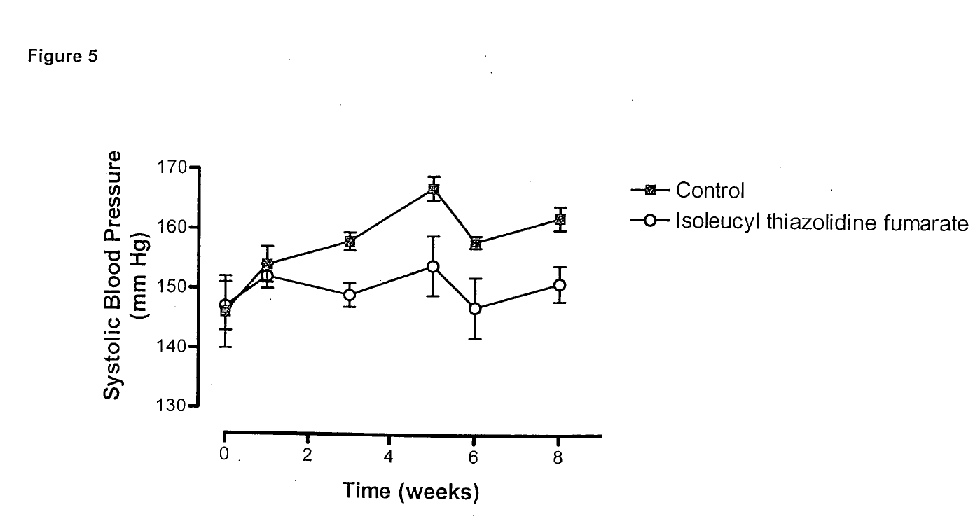

Chronic application of the DPIV-inhibitor isoleucyl thiazolidine fumarate results in the stabilization of systolic blood pressure in the chosen diabetic rat model (FIG. 5).

Animals.

Six pairs of male fatty (fa/fa) VDF Zucker rat littermates were randomly assigned to either a control or treatment (isoleucyl thiazolidine fumarate) group at 440 g body weight (11±0.5 weeks of age). Animals were housed singly, on a 12 hour light/dark cycle (lights on at 6 am) and allowed access to standard rat food, and water ad libitum.

Protocol for Daily Monitoring and Drug Administration.

The treatment group received 10 mg/kg isoleucyl thiazolidine fumarate by oral gavage twice daily (8:00 a.m. and 5:00 p.m.) for 100 days, while the control animals received concurrent doses of vehicle consisting of a 1% cellulose solution. Systolic blood pressure was measured weekly using the tail-cuff procedure.

The test animals (n=5, male Wistar-rats, 200-225 g) initially received 1.5 M Isoleucyl-Thiazolidine in 0.9% saline solution (^{▴}) or the same volume of plain 0.9% saline solution (^{▪}) (control group n=5). The test group additionally obtained an infusion of the inhibitor of 0.75 M/min over 30 min experimental time (*). The control group received during the same time interval an infusion of inhibitor-free 0.9% saline solution. At starting time t=0 all animals were administered an i.d. glucose dose of 1 g/kg 40% dextrose solution (w/v). Blood samples were collected of all test animals in 10 min time intervals. Glucose was analyzed using whole blood (Lifescan One Touch II analyzer) while DPIV-activity and insulin concentration were analyzed in plasma. The insulin radioimmunoassay was sensitive over that range 10 and 160 mU/ml [PEDERSON, R. A., BUCHAN, A. M. J., ZAHEDI-ASH, S., CHEN, C. B. & BROWN, J. C. Reg. Peptides. 3, 53-63 (1982)]. DPIV-activity was estimated spectrophotometrically [DEMUTH, H.-U. and HEINS, J., On the catalytic Mechanism of Dipeptidyl Peptidase IV. in Dipeptidyl Peptidase IV (CD26) in Metabolism and the Immune Response (B. Fleischer, Ed.) R. G. Landes, Biomedical Publishers, Georgetown, 1-35 (1995)]. All data are presented as mean+/−s.e.m.

### Example 18

### Dose Escalation Study in Fatty Zucker Rats After Oral Administration of Glutaminyl Pyrrolidine

Animals:

N=30 male Zucker rats (fa/fa), mean age 11 weeks (5-12 weeks), mean body weight 350 g (150-400 g), were purchased from Charles River (Sulzfeld, Germany).

After delivery they were kept for >12 weeks until nearly all fatty Zucker rats had the characteristics of manifest diabetes mellitus. A group of N=8 animals were recruited for testing three escalating doses of glutaminyl pyrrolidine vs. placebo (saline).

Housing Conditions:

Animals were single-caged under standardized conditions with controlled temperature (22±2° C.) on a 12/12 hours light/dark cycle (light on at 06:00 AM). Sterile standard pelleted chow (ssniff® Soest, Germany) and tap water acidified with HCl were allowed ad libitum.

Catheterization of Carotid Artery:

Fatty Zucker rats of 24-31 weeks (mean: 25 weeks) age, adapted to the housing conditions, were well prepared for the study.

Catheters were implanted into the carotid artery of fatty Zucker rats under general anaesthesia (i.p. injection of 0.25 ml/kg b.w. Rompun® [2%], BayerVital, Germany and 0.5 ml/kg b.w. Ketamin 10, Atarost GmbH & Co., Twistringen, Germany). The animals were allowed to recover for one week. The catheters were flushed with heparin-saline (100 IU/ml) three times per week.

Experimental Design:

Placebo (1 ml saline, 0.154 mol/l) or escalating doses of glutaminyl pyrrolidine (5, 15 and 50 mg/kg b.w.) were administered to groups of N=8 fatty Zucker rats. 375 mg of glutaminyl pyrrolidine were dissolved in 1000 μl DMSO (E. Merck, Darmstadt; Germany [Dimethyl sulfoxide p.a.]). 10 ml saline were added and 1 ml aliquots, each containing 34.09 mg of glutaminyl pyrrolidine, were stored at −20° C. For preparation of the test substance, dose dependent aliquots were diluted in saline.

After overnight fasting, placebo or test substance were administered to the fatty Zucker rats via feeding tube orally (15 G, 75 mm; Fine Science Tools, Heidelberg, Germany) at −10 min An oral glucose tolerance test (OGTT) with 2 g/kg b.w. glucose (40% solution, B. Braun Melsungen, Melsungen, Germany) was administered at ±0 min via a second feeding tube. Venous blood samples from the tail veins were collected at −30 min, −15 min, ±0 min and at 5, 10, 15, 20, 30, 40, 60, 90 and 120 min into 20 μl glass capillaries, which were placed in standard tubes filled with 1 ml solution for blood glucose measurement.

All blood samples were labelled with the following data:

Code number

Animal Number

Date of sampling

Time of sampling

Analytical Methods:

Glucose levels were measured using the glucose oxidase procedure (Super G Glucose analyzer; Dr. Müller Gerätebau, Freital, Germany).

Statistical Methods:

Statistical evaluations and graphics were performed with PRISM® 3.02 (GraphPad Software, Inc.). All parameters were analysed in a descriptive manner including mean and SD.

Effect of Medication on Glucose Tolerance:

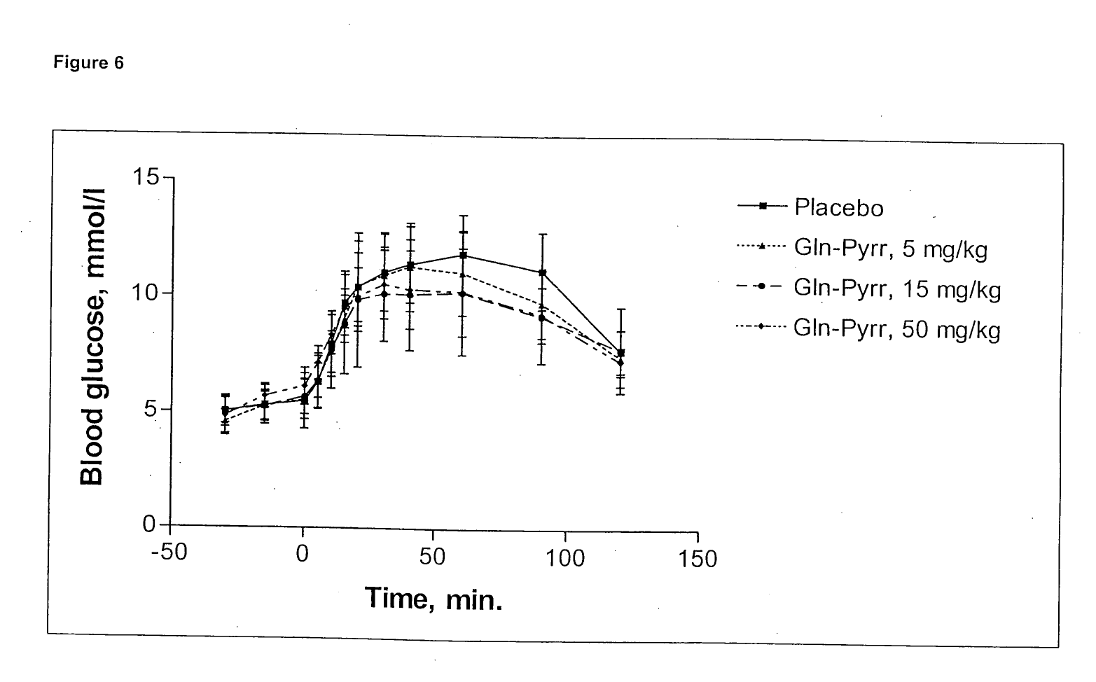

The placebo treated diabetic Zucker rats showed a strongly elevated blood glucose excursion indicating glucose intolerance of manifest diabetes mellitus. Administration of 5 mg/kg b.w. glutaminyl pyrrolidine resulted in a limited improvement of glucose tolerance in diabetic Zucker rats. Significant lowering of elevated blood glucose levels and improvement of glucose tolerance was achieved after administration of 15 mg/kg and 50 mg/kg b.w. glutaminyl pyrrolidine (see FIG. 6).

### Example 19

### Dose Escalation Study in Fatty Zucker Rats After Oral Administration of Glutaminyl Thiazolidine

Animals:

N=30 male Zucker rats (fa/fa), mean age 11 weeks (5-12 weeks), mean body weight 350 g (150-400 g), were purchased from Charles River (Sulzfeld, Germany).

After delivery they were kept for >12 weeks until nearly all fatty Zucker rats had the characteristics of manifest diabetes mellitus. A group of N=8 animals were recruited for testing three escalating doses of glutaminyl thiazolidine vs. placebo (saline).

Housing Conditions:

Animals were single-caged under standardized conditions with controlled temperature (22±2° C.) on a 12/12 hours light/dark cycle (light on at 06:00 AM). Sterile standard pelleted chow (ssniff® Soest, Germany) and tap water acidified with HCl were allowed ad libitum.

Catheterization of Carotid Artery:

Fatty Zucker rats of 24-31 weeks (mean: 25 weeks) age, adapted to the housing conditions, were well prepared for the study.

Catheters were implanted into the carotid artery of fatty Zucker rats under general anaesthesia (i.p. injection of 0.25 ml/kg b.w. Rompun® [2%], BayerVital, Germany and 0.5 ml/kg b.w. Ketamin 10, Atarost GmbH & Co., Twistringen, Germany). The animals were allowed to recover for one week. The catheters were flushed with heparin-saline (100 IU/ml) three times per week.

Experimental Design:

Placebo (1 ml saline, 0.154 mol/l) or escalating doses of glutaminyl thiazolidine (5, 15 and 50 mg/kg b.w.) were administered to groups of N=8 fatty Zucker rats. The respective amounts of glutaminyl thiazolidine were dissolved in 1000 μl saline.

After overnight fasting, placebo or test substance was administered to the fatty Zucker rats via feeding tube orally (15 G, 75 mm; Fine Science Tools, Heidelberg, Germany) at −10 min An oral glucose tolerance test (OGTT) with 2 g/kg b.w. glucose (40% solution, B. Braun Melsungen, Melsungen, Germany) was administered at ±0 min via a second feeding tube. Venous blood samples from the tail veins were collected at −30 min, −15 min, ±0 min and at 5, 10, 15, 20, 30, 40, 60, 90 and 120 min into 20 μl glass capillaries, which were placed in standard tubes filled with 1 ml solution for blood glucose measurement.

All blood samples were labelled with the following data:

Code number

Animal Number

Date of sampling

Time of sampling

Analytical Methods:

Glucose levels were measured using the glucose oxidase procedure (Super G Glucose analyzer; Dr. Möller Gerätebau, Freital, Germany).

Statistical Methods:

Statistical evaluations and graphics were performed with PRISM® 3.02 (GraphPad Software, Inc.). All parameters were analysed in a descriptive manner including mean and SD.

Effect of Medication on Glucose Tolerance:

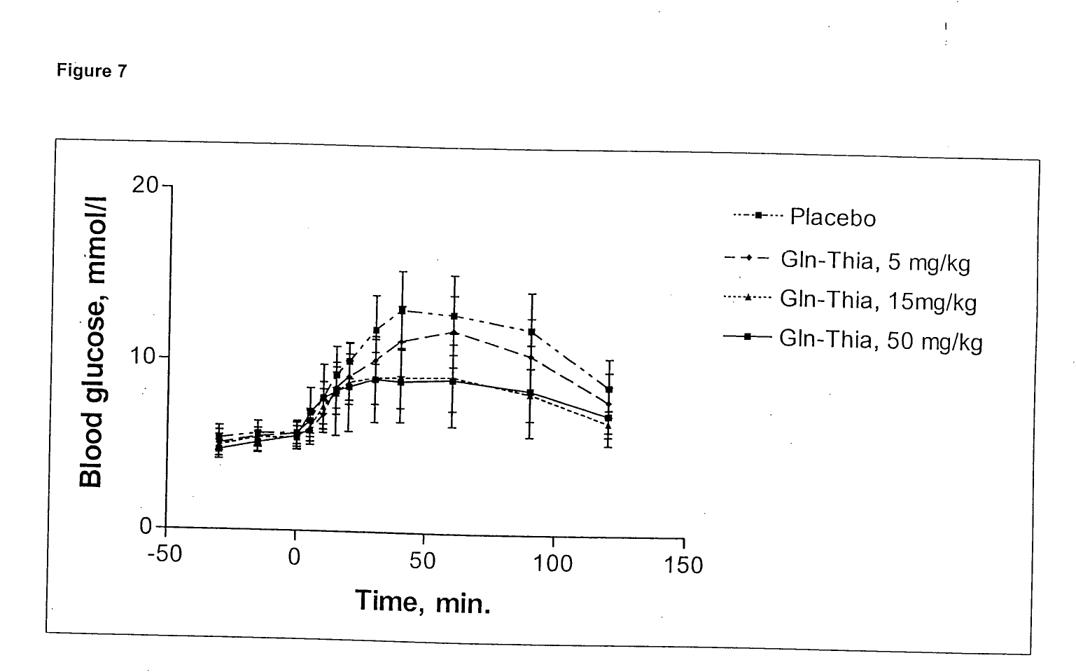

The placebo treated diabetic Zucker rats showed a strongly elevated blood glucose excursion indicating glucose intolerance of manifest diabetes mellitus. Administration of 5 mg/kg b.w., 15 mg/kg and 50 mg/kg b.w glutaminyl thiazolidine resulted in a dose dependent lowering of elevated blood glucose levels and improvement of glucose tolerance in diabetic Zucker rats (see FIG. 7).

### Example 20

### In vivo Inactivation of Glutaminyl Thiazolidine After Oral Administration to Wistar Rats

Animals/Experimental Design:

Glutaminyl thiazolidine was administered to Wistar rats orally as described in example 9.

Analytical Methods:

After application of placebo or glutaminyl thiazolidine, arterial blood samples were taken at 2.5, 5, 7.5, 10, 15, 20, 40, 60 and 120 min from the carotid catheter of the conscious unrestrained rats to determine the formation of degradation products of glutaminyl thiazolidine.

For analysis, simple solid phase extraction procedure on C18 cartridges was used to isolate the compounds of interest from the plasma. The extracts were analysed using reversed-phase liquid chromatography on Lichrospher 60 RP Select B column hyphenated with tandem mass spectrometry operating in the APCI positive mode. An internal standard method was used for quantification.

Results:

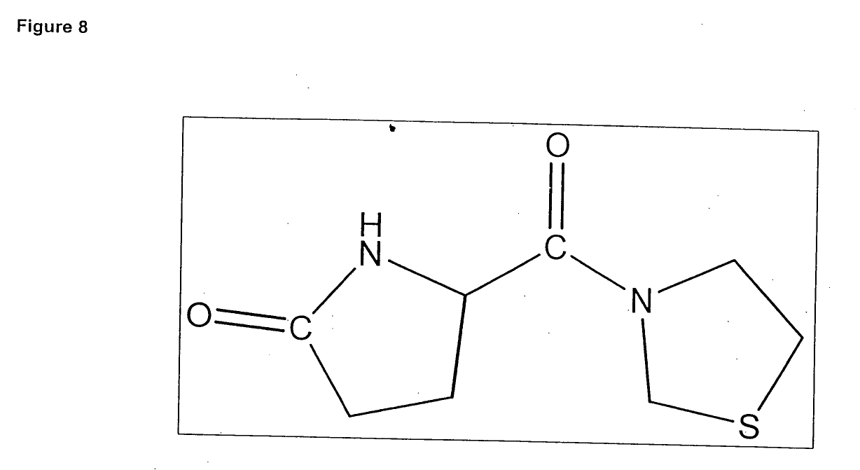

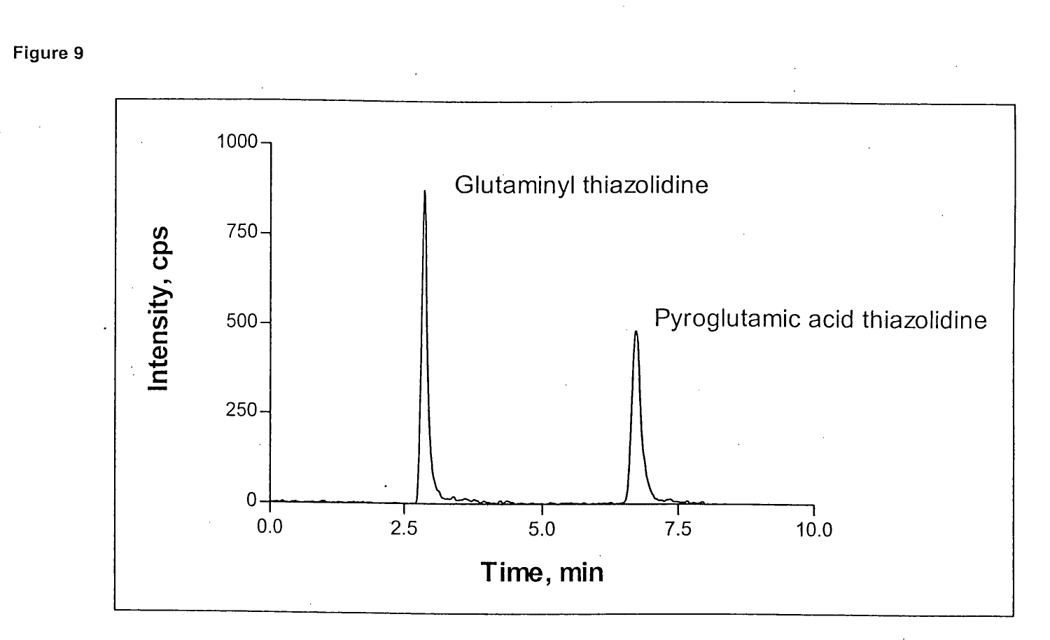

After oral administration of glutaminyl thiazolidine to Wistar rats, a degradation of the compound was found. Using LC/MS, the degradation product could be defined as pyroglutaminyl thiazolidine. See FIGS. 8 and 9.

We claim:

1. Use of at least one inhibitor of dipeptidyl peptidase IV (DPIV) or DPIV-like enzyme activity for the preparation of a pharmaceutical composition for lowering blood pressure levels or related disorders in a mammal.

2. The use according to claim 1, wherein the inhibitor is selected from the group consisting of dipeptide compounds, peptide compounds comprising tri-, tetra- and pentapeptides, peptidylketones, aminoketone derivatives and side chain modified DPIV inhibitors.

3. The use according to claim 1, wherein the dideptidyl peptidase IV-like enzyme is selected from the group consisting of fibroblast activation protein α, dipeptidyl peptidase IV β, dipeptidyl aminopeptidase-like protein, N-acetylated α-linked acidic dipeptidase, quiescent cell proline dipeptidase, dipeptidyl peptidase II, attractin and dipeptidyl peptidase IV related protein (DPP 8), dipeptidyl peptidase 9 (DPP9), DPRP1, DPRP2, DPRP3 or KIAA1492.

4. The use according to claim 1, wherein the structure of the dideptidyl peptidase IV-like enzyme is undiscovered.

5. The use according to claim 1, wherein the inhibitor is a dipeptide-like compound formed from an amino acid and a thiazolidine or pyrrolidine group, and salts thereof.

6. The use according to claim 5 wherein the dipeptide compound is selected from the group consisting of L-threo-isoleucyl pyrrolidine, L-allo-isoleucyl thiazolidine, L-threo-isoleucyl pyrrolidine L-allo-isoleucyl pyrrolidine, L-glutaminyl thiazolidine, L-glutaminyl pyrrolidine, L-glutamic acid thiazolidine, L-glutamic acid pyrrolidine, alanyl pyrrolidine, N-valyl prolyl-O-benzoyl hydroxylamine and salts thereof.

7. The use according to claim 1, wherein the inhibitor is a peptide compound useful for competitive modulation of dipeptidyl peptidase IV catalysis represented by the general formula 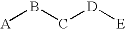
wherein
A is an amino acid except a D-amino acid;
B is an amino acid selected from Pro, Ala, Ser, Gly, Hyp, acetidine-(2)-carboxylic acid and pipecolic acid,
C is any amino acid except Pro, Hyp, acetidine-(2)-carboxylic acid, pipecolic acid and except N-alkylated amino acids, e.g. N-methyl valine and sarcosine,
D is any amino acid or missing, and
E is any amino acid or missing,
or:
C is any amino acid except Pro, Hyp, acetidine-(2)-carboxylic acid, pipecolic acid, except N-alkylated amino acids, e.g. N-methyl valine and sarcosine and except a D-amino-acid;
D is any amino acid selected from Pro, Ala, Ser, Gly, Hyp, acetidine-(2)-carboxylic acid and pipecolic acid, and
E is any amino acid except Pro, Hyp, acetidine-(2)-carboxylic acid, pipecolic acid and except N-alkylated amino acids, e.g. N-methyl valine and sarcosine.

8. The use according to claim 1, wherein the inhibitor is a peptidylketone represented by the general formula 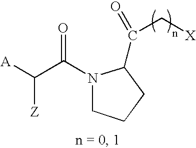
including all stereoisomers and pharmaceutically by acceptable salts thereof,
wherein
A is selected from 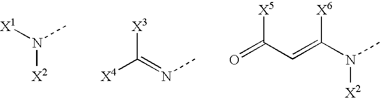
and
X¹ is H or an acyl or oxycarbonyl group or an amino acid or peptide residue,
X² is H, —(CH)ₙ—NH—C₅H₃N—Y with n=2-4 or C₅H₃N—Y (a divalent pyridyl residue) and Y is selected from H, Br, Cl, I, NO₂ or CN,
X³ is H or a phenyl or pyridyl residue, unsubstituted or substituted with one, two or more alkyl, alkoxy, halogen, nitro, cyano or carboxy residues,
X⁴ is H or a phenyl or pyridyl residue, unsubstituted or substituted with one, two or more alkyl, alkoxy, halogen, nitro, cyano or carboxy residues,
X⁵ is H or an alkyl, alkoxy or phenyl residue,
X⁶ is H or an alkyl residue.
for n=1
X is selected from: H, OR², SR², NR²R³, N⁺R²R³R⁴, wherein:
R² stands for acyl residues, which are unsubstituted or substituted with one, two or more alkyl, cycloalkyl, aryl or heteroaryl residues, or for all amino acids and peptidic residues, or alkyl residues, which are unsubstituted or substituted with one, two or more alkyl, cycloalkyl, aryl and heteroaryl residues,
R³ stands for alkyl and acyl functions, wherein R² and R³ may be part of one or more ring structures of saturated and unsaturated carbocyclic or heterocyclic structures,
R⁴ stands for alkyl residues, wherein R² and R⁴ or R³ and R⁴ may be part of one or more ring structures of saturated and unsaturated carbocyclic or heterocyclic structures,
for n=0
X is selected from: 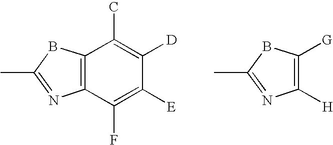
wherein
B stands for: O, S, NR⁵, wherein R⁵ is H, an alkyliden or acyl,
C, D, E, F, G, H are independently selected from unsubstituted and substituted alkyl, oxyalkyl, thioalkyl, aminoalkyl, carbonylalkyl, acyl, carbamoyl, aryl and heteroaryl residues; and
for n=0 and n=1
z is selected from H, or a branched or single chain alkyl residue from C₁-C₉ or a branched or single chain alkenyl residue from C₂-C₉, a cycloalkyl residue from C₃-C₈, a cycloalkenyl residue from C₅-C₇, an aryl- or heteroaryl residue, or a side chain selected from all side chains of all natural amino acids or derivatives thereof.

9. The use according to claims 1, wherein the inhibitor is an aminoketone derivative represented by the general formulas 5, 6, 7, 8, 9, 10 and 11, including all stereoisomers and pharmaceutical acceptable salts thereof, 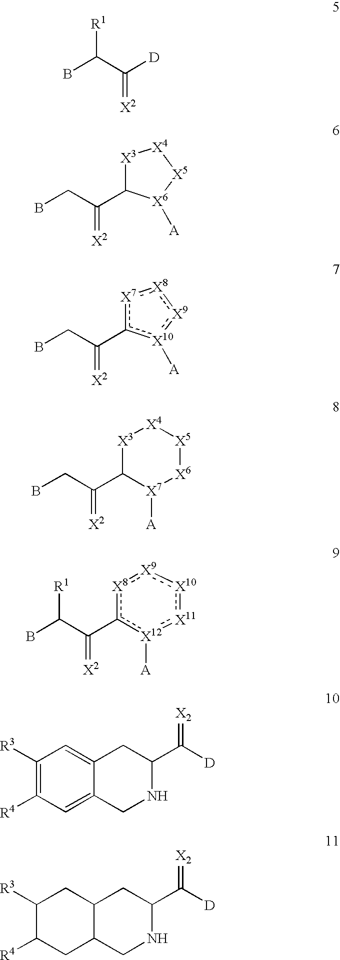
wherein:
R¹ is H, a branched or linear C₁-C₉ alkyl residue, a branched or linear C₂-C₉ alkenyl residue, a C₃-C₅ cycloalkyl-, C₅-C₇ cycloalkenyl-, aryl- or heteroaryl residue or a side chain of a natural amino acid or a derivative thereof;
R³ and R⁴ are independently selected from H, hydroxy, alkyl, alkoxy, aryloxy, nitro, cyano or halogen,
A is H or an isoster of an carbonic acid, like a functional group selected from CN, SO₃H, CONHOH, PO₃R⁵R⁶, tetrazole, amide, ester, anhydride, thiazole and imidazole;
B is selected from: 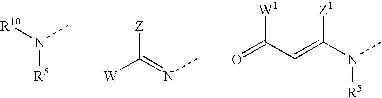
wherein:
R⁵ is H, —(CH)ₙ—NH—C₅H₃N—Y with n=2-4 and C₅H₃N—Y (a divalent pyridyl residue) with Y═H, Br, Cl, I, NO₂ or CN,
R¹⁰ is H, an acyl, oxycarbonyl or a amino acid residue
W is H or a phenyl or pyridyl residue, unsubstituted or substituted with one, two or more alkyl, alkoxy, halogen, nitro, cyano or carboxy residues,
W¹ is H, an alkyl, alkoxy or phenyl residue,
Z is H or a phenyl or pyridyl residue, unsubstituted or substituted with one, two or more alkyl, alkoxy, halogen, nitro, cyano or carboxy residues,
Z¹ is H or an alkyl residue,
D is a cyclic C₄-C₇ alkyl, C₄-C₇ alkenyl residue which can be unsubstituted or substituted with one, two or more alkyl groups or a cyclic 4-7-membered heteroalkyl or a cyclic 4-7-membered heteroalkenyl residue,
X² is O, NR⁶, N⁺(R⁷)₂, or S,
X³ to X¹² are independently selected from CH₂, CR⁸R⁹, NR⁶, N⁺(R⁷)₂, O, S, SO and SO₂, including all saturated and unsaturated structures,
R⁶, R⁷, R⁸, R⁹ are independently selected from H, a branched or linear C₁-C₉ alkyl residue, a branched or linear C₂-C₉ alkenyl residue, a C₃-C₈ cycloalkyl residue, a C₅-C₇ cycloalkenyl residue, an aryl or heteroaryl residue,
with the following provisos:
Formula 6: X⁶ is CH if A is not H,
Formula 7: X¹⁰ is C if A is not H,
Formula 8: X⁷ is CH if A is not H,
Formula 9: X¹² is C if A is not H.

10. The use according to claim 1, wherein the inhibitor of DPIV or DPIV-like enzyme activity is represented by the general formula, 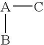
including all stereoisomers and pharmaceutical acceptable salts thereof,
wherein
A is an amino acid having at least one functional group in the side chain,
B is a chemical compound covalently bound to at least one functional group of the side chain of A, especially
an oligopeptide having a chain length of up to 20 amino acids, or
a polyethylene glycol having a molar mass of up to 20 000 g/mol,
an optionally substituted organic amine, amide, alcohol, acid or aromatic compound having from 8 to 50 C atoms and
C is a thiazolidine, pyrrolidine, cyanopyrrolidine, hydroxyproline, dehydroproline or piperidine group amide-bound to A.

11. The use according to claim 10, wherein A is an amino acid, preferably an α-amino acid, especially a natural α-amino acid having at least one functional group in the side chain selected from the group consisting of threonine, tyrosine, serine, arginine, lysine, aspartic acid, glutamic acid or cysteine.

12. The use according to claim 1, wherein said inhibitor is a pharmaceutical composition comprising a pharmaceutically acceptable carrier or diluent and a therapeutically effective amount of a said inhibitor or a pharmaceutically acceptable acid addition salt thereof.

13. The use according to claim 1, wherein said inhibitor or said inhibitors are used in combination with a pharmaceutically acceptable carrier and/or diluent.

14. The use according to claim 1, wherein said at least one inhibitor is administered in multiple administrations.

15. The use according to claim 1, wherein the mammal demonstrates clinically inappropriate basal and post-prandial hyperglycemia or blood pressure levels or both.

16. The use according to claim 1 the prevention or alleviation of pathological abnormalities of metabolism of mammals such as glucosuria, hyperlipidaemia, metabolic acidosis and diabetes mellitus resulting in lowered blood pressure.

17. The use according to claim 1 for lowering blood pressure levels in mammals experiencing blood pressures in excess of 140 mm Hg, wherein the at least one inhibitor is administered periodically.

18. The use according to claim 1 comprising the oral administration of the at least one inhibitor or pharmaceutical composition.
